(19) United States
(12) Patent Application Publication
(10) Pub. No.: US 2010/0240025 A1
(43) Pub. Date: Sep. 23, 2010

(54) Bacteriophage with Enhanced Lytic Activity

(76) Inventors: Timothy Read, Atlanta, GA (US); Shanmuga Sozhamannan, Timonium, MD (US)

Correspondence Address: MORGAN LEWIS & BOCKIUS LLP, 1111 PENNSYLVANIA AVENUE NW, WASHINGTON, DC, 20004, US

(21) Appl. No.: 12/664,759

(22) PCT Filed: Jun. 16, 2008
(86) PCT No.: PCT/US08/67089

§ 371 Date: Jun. 7, 2010

(30) Foreign Application Priority Data
Jun. 15, 2007 (US) 60944130

(51) Int. Cl. C12Q 1/70; C12N 7/01; C07H 21/00; C07K 14/00

(52) U.S. Cl. 435/5; 435/235.1; 536/23.1; 530/350; 530/324

(57) ABSTRACT

This invention encompasses an isolated Bacillus phage AP50 that has one or more nucleotide substitutions in the phage genome, whereby the one or more nucleotide substitutions increase lytic activity of the phage. In addition, the invention encompasses proteins expressed by the phages, compositions containing the proteins and/or the phage as well as methods of using the Bacillus phage AP50 to test for the presence of B. anthracis.

### RELATED APPLICATIONS

This application claims priority to U.S. Provisional Application 60/944,130 (filed on Jun. 15, 2007) which is incorporated by reference in its entirety.

### GOVERNMENT SUPPORT

The present invention arose in part from research funded by the Defense Threat Reduction Agency, Department of Defense. The Government has certain rights in the invention.

### BACKGROUND OF THE INVENTION

Bacillus anthracis, a category A biothreat agent, is a spore forming Gram-positive bacterium of the Bacillus cereus sensu lato group. It is a zoonotic soil bacterium that infects animals and occasionally humans causing the disease anthrax. Bacillus anthracis are aerobic and spore-forming bacilli.

The notoriety of B. anthracis stems from the fact that it was successfully used in bioterror attacks via mail laced with anthrax spores, following the 9/11 terrorist attacks. The prospect of biothreats using B. anthracis and the possibility of naturally emergent or deliberately created antibiotic resistant B. anthracis, calls for highly integrated and enhanced technological platforms, capable of specifically targeting and rapidly screening for this organism. This need is best illustrated in case of a bacterial bioterror attack where timely detection and intervention with countermeasures such as antibiotic therapy are paramount in preventing fatal consequences.

Pathology due to B. anthracis infection is primarily due to the release by the organism of “protective antigen” (PA) in association with lethal factor (LF) and edema factor (EF) (Sellman et al. (2001) Science 292: 695-7). The complete DNA and protein sequence of PA has been published and its three-dimensional structure is known from x-ray crystallography (Petosa et al. (1997) Nature 385: 833-8). The characteristics and biological functions of the four domains of PA are also available permitting selection of epitopes within the domains based on antigenic properties (Petosa et al.; Little et al. (1996) Microbiology 142: 707-15; Brossier et al. (1999) Infect. Immun 67: 964-7; Brossier et al. (2000) Infect. Immun. 68: 1781-6; Mogridge et al. (2001) J. Bacteriol. 183: 2111-6). In animal studies, as well as studies of natural human infection, it was shown that individuals who survived an infection produced antibodies to PA suggesting its importance in protection (Brachman (1962) Am. J. Public Health 52: 632-45).

Bacillus anthracis is closely related to other members of the B. cereus group of bacteria. Laboratory isolates can generally be distinguished either by polymerase chain reaction (PCR) amplification of toxin genes and plasmids (pXOI and pXO2) and by other clinical laboratory analysis, especially if toxin genes are not present. An isolate of B. anthracis typically appears as a white or gray colony that is nonhemolytic or, at most, weakly hemolytic, nonmotile, and is penicillin susceptible. The ability to form capsule is also diagnostic and is typically demonstrated after culture on nutrient agar containing 0.7% sodium bicarbonate incubated overnight under CO₂. Colonies of the capsulated B. anthracis appear mucoid and the capsule can be visualized by staining with M'Fadyean polychrome methylene blue or India ink. An additional important evaluation is also the susceptibility to gamma phage, a bacteriophage.

Bacteriophages have been and still remain useful tools for bacterial species and strain differentiation (Hagens and Loessner (2007) Appl. Microbiol. Biotechnol. 76:513-9; McAuliffe et al. (2007) p. 1-42. In Mc Grath and van Sinderen (eds.), Bacteriophage. Genetics and Molecular Biology Caister Academic Press; McKinstry and Edgar (2005) p. 430-440. In Waldor et al. (eds.), Phages: their role in bacterial pathogenesis and biotechnology ASM press; Petty et al. (2007) Trends Biotechnol. 25:7-15) although evidence for successful application of phage therapy is still sparse in western medicine (Sulakvelidze et al. (2001) Antimicrob. Agents Chemother. 45:649-59).

Recently, the inherent binding specificity and lytic action of bacteriophage encoded enzymes called lysins have been exploited for the rapid detection and killing of B. anthracis (Schuch et al. (2002) Nature 418:884-9). It was demonstrated that the PlyG lysin, isolated from the γ phage of B. anthracis, specifically kills B. anthracis isolates and other members of the B. anthracis ‘cluster’ of bacilli in vitro and in vivo. Both vegetative cells and germinating spores were shown to be susceptible. The lytic specificity of PlyG was also exploited as part of a rapid method for the identification of B. anthracis thus indicating that PlyG is a tool for the treatment and detection of B. anthracis (Schuch et al. (2002) Nature 418:884-9).

A well-known B. anthracis specific phage of the Tectiviridae family, AP50, was first isolated from soil in 1972 using B. anthracis Sterne as the host (Ackermann et al. (1978) Can. J. Microbiol. 24:986-93; Nagy, E. (1974) Acta. Microbiol. Acad. Sci. Hung. 21:257-63). Originally it was thought to be an RNA phage, but later shown to contain double stranded (ds) DNA and phospholipid (Nagy et al. (1976) J. Gen. Virol. 32:129-32). AP50 was also shown to have a narrow host range; only one third of the 34 B. anthracis strains and none of the 52 strains belonging to 6 different Bacillus spp were susceptible to infection by AP50 (Nagy et al. (1977) J. Gen. Microbiol. 102:215-9). Nine major structural proteins were identified on SDS-PAGE gels. The molecular weight of the phage DNA was estimated to be 9×10⁶ daltons (Nagy et al. (1982) J. Gen. Virol 62:323-329). Treatment with organic solvents such as chloroform (5%) and ether (25%) for 30 minutes inactivated the phage to a survival of about 1×10⁻⁴ (Nagy and Ivanovics (1982) Acta. Microbiol. Acad. Sci. Hung. 29:89-98).

Virions of the Tectiviridae family of phages possess isometric nucleocapsids with icosahedral symmetry and a capsid shell composed of two layers: a smooth, rigid 3 nm thin outer shell and a flexible, 5-6 nm thick inner lipoprotein vesicle. Virions contain one molecule of linear double stranded DNA with a total genome length of ˜15 kb containing inverted terminal repeats (ITRs). A protein essential for the proposed protein primed DNA replication process of the phage is bound to the termini of the linear molecule (ICTV. 2002. International committee on taxonomy of viruses-ICTVdB descriptions: 68. Tectiviridae). While phage PRD1, infecting Gram-negative bacteria carrying Inc P, N, W plasmids, is considered to be a model phage for this family (Grahn et al. (1994) J. Bacteriol. 176:3062-8; Saren et al. (2005) J. Mol. Biol. 350:427-40), several phages belonging to this family have also been isolated in Gram-positive bacteria; e.g., AP50, Bam35, Gi101, Gi116, and NS11 (Nagy and Ivanovics (1982) Acta. Microbiol. Acad. Sci. Hung. 29:89-98; Ravantti et al. (2003) Virology 313:401-14; Verheust et al. (2005) J. Bacteriol. 187:1966-73; Verheust et al. (2003) Microbiology 149:2083-92).

Among these, phages Bam35, Gil01 and Gil16 have been genetically characterized and their genome sequences have been determined (Ravantti et al. (2003), Verheust et al. (2005); Verheust et al. (2003); Stromsten et al. (2003) J. Bacteriol. 185:6985-9). These genomes exhibit a high degree of similarity in genetic organization to a linear plasmid found in B. cereus ATCC 14579, pBclin15 (Ivanova et al. (2003) Nature 423:87-91). Unlike many temperate phages whose genomes are integrated into the host chromosome, some members of this family of phages exist as extra-chromosomal linear plasmids in the lysogenic state. The linear ends are protected from nucleolytic attacks by proteins (Stromsten et al. (2003)).

Although Gram-negative bacteria infecting phage PRD1 and Gram-positive bacterium phage Bam35 have closely related virion morphology and genome organization, they have no detectable sequence similarity. There is strong evidence that the Bam35 coat protein has the “double-barrel trimer” arrangement of PRD1 that was first observed in adenovirus and is predicted to occur in other viruses with large facets. It has been suggested that this group includes viruses infecting very different hosts in all three domains of life: eucarya, bacteria and archaea suggesting a single viral lineage for this very large group of viruses (Saren et al. (2005) J. Mol. Biol. 350:427-40).

The standard diagnostic tests for suspected B. anthracis, recommended by the Centers for Disease Control and Prevention (CDC) include several procedures. Presumptive identification to genus level (Bacillus family of organisms) requires Gram stain and colony identification and presumptive identification to species level (B. anthracis) requires tests for motility, lysis by γ phage, capsule production and visualization, hemolysis, wet mount and malachite green staining for spores. Confirmatory identification of B. anthracis may include lysis by γ phage, capsular staining, and direct fluorescent antibody (DFA) testing on capsule antigen and cell wall polysaccharide. Thus, testing for γ phage sensitivity has been an integral part of B. anthracis identification (CDC (2002) Center for disease control and prevention: Anthrax Q & A: Diagnosis). γ phage exhibits a fairly narrow host range but several B. cereus strains (e.g., ATCC 4342) have been shown to be sensitive to infection by this phage (Abshire et al. (2005) J. Clin. Microbiol. 43:4780-8, Brown et al. (1955) J. Infect. Dia 0.96:34-9; Davison et al. (2005) J. Bacteriol. 187:6742-9; Schuch et al. (2002) Nature 418:884-9). Several phages (CP51, CP54 and TP21) isolated from B. cereus and B. thuringiensis strains have been successfully used for transducing chromosomal markers and plasmids between B. anthracis strains (Green et al. (1985) Infect Immun 49:291-7; Ruhfel et al. (1984) J. Bacteriol. 157:708-11; Thome, C. B. (1968) Bacteriol. Rev. 32:358-61; 37; Walter and Aronson (1991) Appl. Environ. Microbiol. 57:1000-5; Yelton and Thorne (1970) J. Bacteriol. 102:573-9). However, their utility as B. anthracis diagnostic phages is limited because of their broad host range.

### SUMMARY OF THE INVENTION

This invention provides for an isolated Bacillus phage AP50 that has one or more nucleotide substitutions in the phage genome, whereby the one or more nucleotide substitutions increase lytic activity of the phage. The invention encompasses all 31 genes (ORF1-31) which make up the genome and the proteins encoded by these genes. In addition, the invention provides for methods of using the phage to test for the presence of B. anthracis.

In one embodiment of the invention, the isolated Bacillus phage AP50 has a nucleotide substitution at a position corresponding to nucleotide 271 of SEQ ID NO: 55 (nucleotide 271 of ORF28). Preferably, the substitution at nucleotide 271 is a C to T substitution. In another embodiment, the isolated Bacillus phage AP50 has a position corresponding to nucleotide 154 of SEQ ID NO: 63 (such as e.g. a T to C substitution).

The Bacillus phage may have the nucleotide sequence of SEQ ID NO: 63. In another embodiment, the Bacillus phage AP50 has the nucleotide sequence of SEQ ID NO: 6 and nucleotide substitutions including a nucleotide substitution at a positions corresponding to nucleotides at position 154 and 12,881 (271 of SEQ ID NO: 55 (nucleotide 271 of ORF28)) of SEQ ID NO: 63.

The isolated Bacillus phage AP50 according to the invention comprises various genes which are encoded by various open reading frames. In one embodiment, the Bacillus phage genome comprises the nucleotide sequence of one or more of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61 or the complement thereof.

The isolated Bacillus phage AP50 may be part of a composition, including but not limited to pharmaceutical compositions, and a kit. In one embodiment, the phage is in a composition or kit which also contains gamma phage.

The invention further provides for nucleic acids from the isolated Bacillus AP50 phage. In one embodiment, the isolated nucleic acids encode protein having the amino acid sequence of any of SEQ ID NO: 2, 4, 6, 8, 10, 12, 14, 16, 18, 20, 22, 24, 26, 28, 30, 32, 34, 36, 38, 40, 42, 44, 46, 48, 50, 52, 56, 58, 60, or 62 (i.e. amino acid sequences of ORF1 to ORF31). In another embodiment, the nucleic acids comprises any of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, or 61. In yet another embodiment of the invention, the isolated nucleic acid has at least 85% sequence identity to any of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, or 61. In an alternate embodiment, the isolated nucleic acid contains SEQ ID NO: 63. The invention also provides for recombinant phages comprising any of the nucleic acids. In a preferred embodiment, the recombinant phage comprises SEQ ID NO: 63.

The invention further provides for isolated proteins from an isolated Bacillus phage AP50 that has one or more nucleotide substitutions in the phage genome, whereby the one or more nucleotide substitutions increase lytic activity of the phage. In one embodiment, the isolated proteins comprises the amino acid sequence of any of 2, 4, 6, 8, 10, 12, 14, 16, 18, 20, 22, 24, 26, 28, 30, 32, 34, 36, 38, 40, 42, 44, 46, 48, 50, 52, 56, 58, 60, or 62 (i.e. the amino acid sequences of ORF1 to ORF31). In another embodiment of the invention, the isolated protein has at least 85% sequence identity to any of SEQ ID NO: 2, 4, 6, 8, 10, 12, 14, 16, 18, 20, 22, 24, 26, 28, 30, 32, 34, 36, 38, 40, 42, 44, 46, 48, 50, 52, 56, 58, 60, or 62.

The invention also provides for methods of detecting the presence of B. anthracis. One embodiment of the invention is a method for detecting the presence of B. anthracis in a subject that has at least the steps of (a) isolating a biological sample from the subject, (b) contacting a sample with a phage according to the invention (i.e. Bacillus phage AP50 that has one or more nucleotide substitutions in the phage genome, whereby the one or more nucleotide substitutions increase lytic activity of the phage) and (c) detecting for the presence of bacterial lysis. In this method, the increased presence of bacterial lysis compared to a control indicates the presence of B. anthracis in the sample. The step of isolating the biological sample may also encompass incubating biological sample under conditions sufficient to induce growth of B. anthracis. In one embodiment, the control is a sample which does not contain B. anthracis. In another embodiment, the contacting is carried out under conditions sufficient to induce phage lysis of B. anthracis. The method may also further comprise contacting the biological sample with gamma phage prior to detecting for the presence of bacterial lysis.

### BRIEF DESCRIPTION OF THE DRAWINGS

The foregoing summary, as well as the following detailed description of the invention, be better understood when read in conjunction with the appended figures. For the purpose of illustrating the invention, shown in the figures are embodiments of the present invention. It should be understood, however, that the invention is not limited to the precise arrangements, examples and instrumentalities shown.

FIG. 1 shows the plaque morphology of (a) mixed lysate and (b) AP50c plaques after overnight incubation at room temperature and at 37° C.

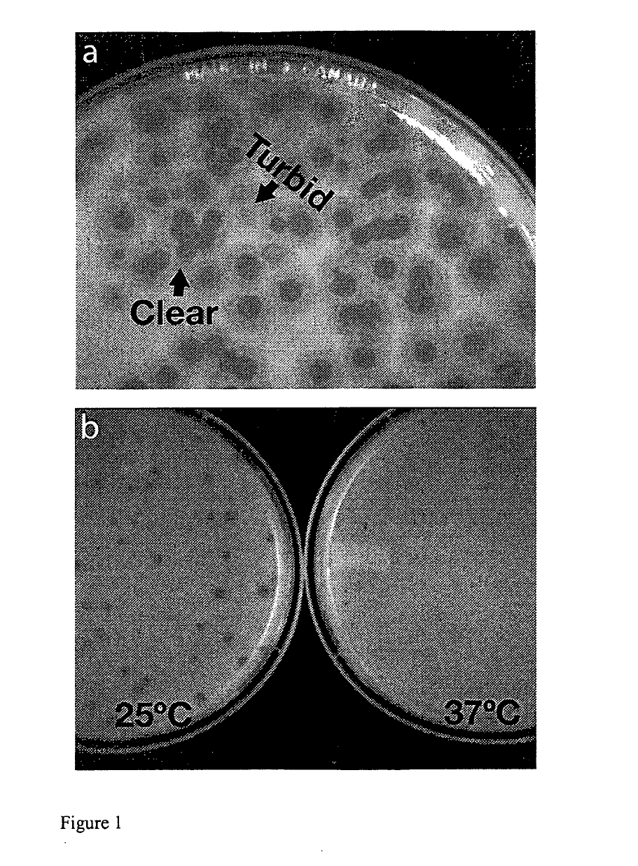

FIG. 2 shows Transmission electron micrographs of AP50 phage particles. FIG. 2A shows Uranyl acetate staining at a magnification of 297K. FIGS. 2B and 2C show AP50 after phosphotungstate staining at a magnification of 297K. Specifically, FIGS. 2B and 2C show damaged particles (chloroform treatment) after removal of the protein capsid. The inner lipoprotein vesicles and a tail-like tube derived from this vesicle are seen. The scale bar in the figures is 100 nm.

FIG. 3 shows various features of the AP50 genome. FIG. 3A shows the genome map of AP50. Three clusters of genes based on functional grouping and similarities to other tectiviral phages are shown. ORF boxes are color coded to indicate the degree of amino acid identity with proteins of other tectiviral phages. The ORFs have between <15% to 80% amino acid identity with proteins of other tectiviral phages. ITR: inverted terminal repeat; HVR: highly variable region. Open arrow heads indicate the locations of the mutations in AP50c phages. FIG. 3B shows a visualization summary of whole-genome nucleotide alignments of Gram-positive tectiviral phages. The ClustalW alignment file generated from multifasta alignment was visualized in Base by Base (Brodie et al. 2004, BMC Bioinformatics 5:96) In this type of alignment, if two sequences have insertions or deletions relative to one another, the output looks different depending on which of the two sequences is used as the base sequence. White, perfect nucleotide homology; blue, SNP; red, deletions in the indicated phage; green, insertions in the indicated phage. The genbank accession numbers for the sequences used in the alignment are: Bam35c (NC_{—}005258), pBth35646 (NZ_AAJM00000000), Gi101 (AJ536073), Gil16c (AY701338), AP50 (EU408779), pBclin15 (AE01878). FIG. 3C shows the sequence changes in AP50c and AP50t genomes. The mutation in the non coding region just upstream of ORF-1 at nt position 164 is indicated. The second mutation is in ORF 28 at position 12,881 and changes the amino acid residue 91 (an isoleucine in AP50c to a valine in AP50t).

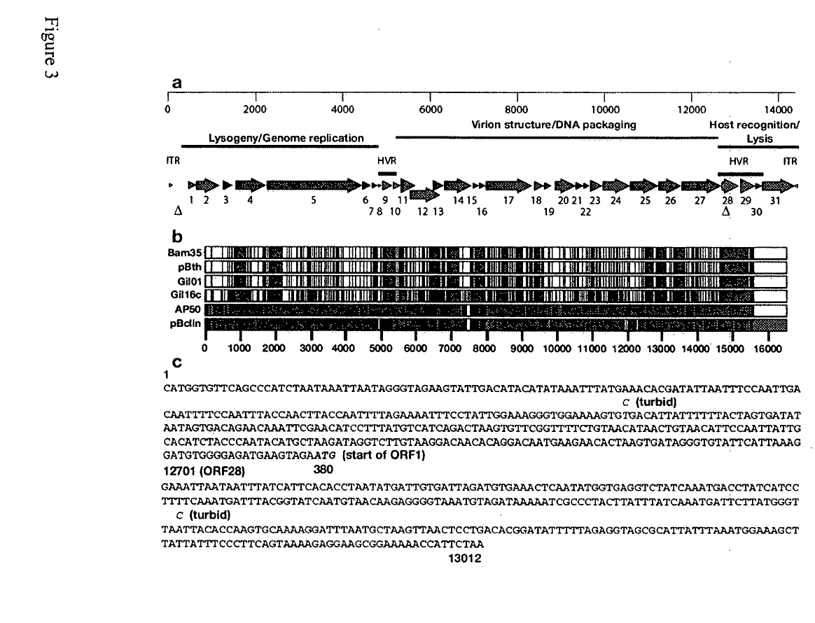

FIG. 4 shows ClustalW alignment of amino acid of ORF31 with similar ORFs in Gil16c (ORF31), Bam35 (ORF31) and pBClin15 (ORF28) genomes.

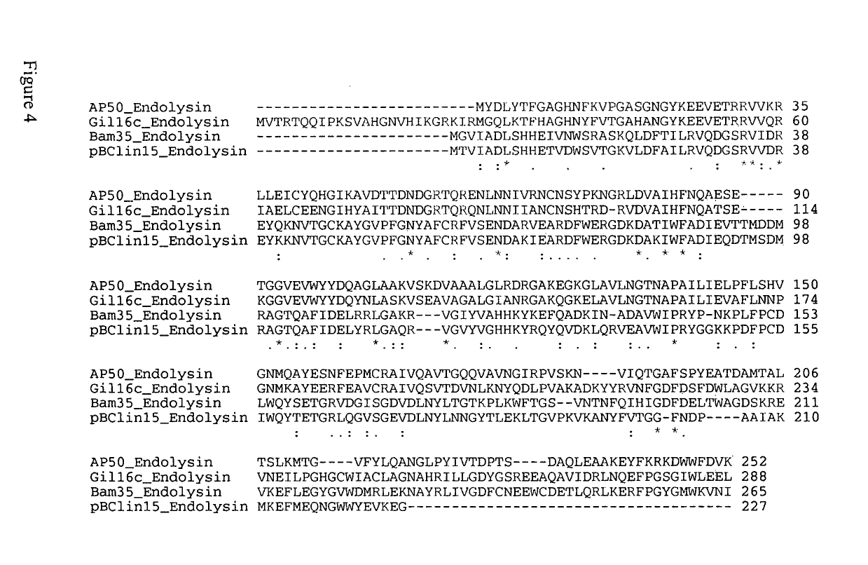

FIG. 5 shows the colony morphologies of B. anthracis Sterne strain 34F₂ after infection with AP50c or AP50t. FIG. 5A shows uninfected 34F₂ cells diluted and plated on phage assay agar plates. FIG. 5B shows AP50t infected culture, diluted and plated; FIGS. 5C and 5D show AP50 t and AP50c infected cultures, respectively, plated on phage assay agar plates.

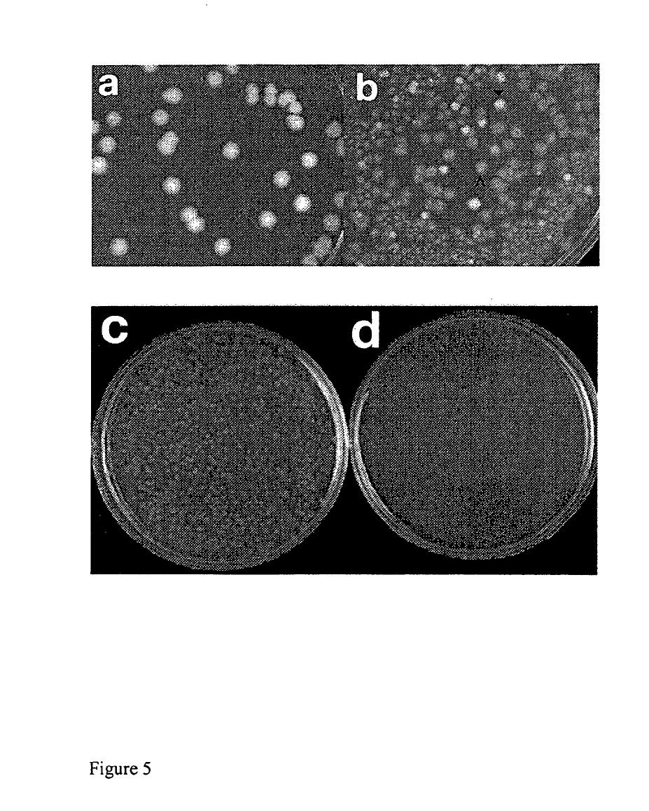

FIG. 6 shows the morphology of AP50c resistant 34F₂ mutants. FIG. 6A depicts logarithmically grown cultures were incubated statically at room temperature overnight. Wild type 34F₂ cells settled at the bottom of the culture tube as a pellet and the AP50^{R} mutant contained a viscous material which prevented cell settling at the bottom of the tube. FIG. 6B is a scanning electron micrographs of wild type 34F₂ infected with AP50. The arrows indicate the AP50 particels attached to the outer surface of the bacterium. FIG. 6C is a scanning electron micrograph of 34F₂ AP50^{R} mutant infected with AP50 showing the presence of polysaccharide material coating the outer cell surface and absence of attached phage particles.

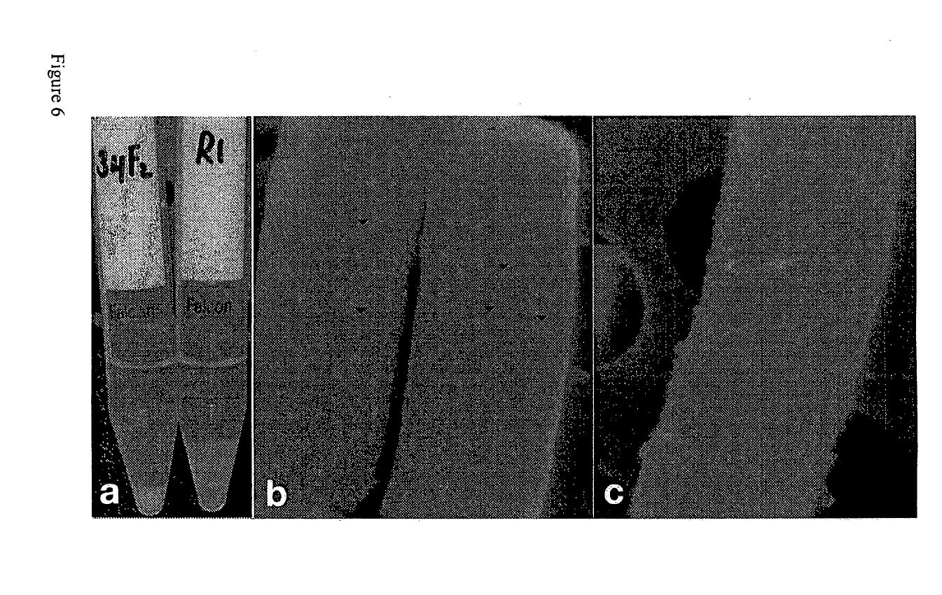

### DETAILED DESCRIPTION

### General Description

The inventors have isolated and characterized the genome of a B. anthracis specific phage of the Tectiviridae family, AP50 (herein after referred to as “AP50 phage” throughout the specification and claims). Thus, the invention encompasses all 31 genes (ORF1-31) which make up the genome and the proteins encoded by these genes. In addition, the invention encompasses a variant of AP50 which exhibits increased lytic activity.

The present invention provides AP50 phages or parts thereof that inhibit growth of target bacteria (e.g., B. anthracis) because of their increased bacterio-lytic properties. The phages are thus useful for inhibiting bacterial growth or presence in the environment and for treating bacterial infection in a subject in need of such treatment. In some embodiments, the AP50 phage are unable to replicate in a target bacteria and yet inhibit the growth of the target bacteria, they can be administered as a defined dose therapeutic composition for treatment of bacterial infections. This provides substantial regulatory advantages, which prevent changing stoichiometric ratios of treatment and target entities as the bacterial infection and bacteriophage replication processes progress.

This invention provides that, for each pathogenic bacteria target (e.g., B. anthracis), phage from the Tectiviridae family, including AP50, will be useful as a defined dose therapeutic agent to inhibit growth of or kill B. anthracis.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

AP50 Phage with Enhanced Lytic Activity

As used herein “bacteriophage” is generally shortened to “phage” as is well known in the art. Bacteriophage typically refers to a functional phage, but in many contexts herein may refer to a part thereof, generally exhibiting a particular function. The AP50 phage is modified as such to have enhanced and/or increased lytic properties. In some circumstances, the term may also refer to portions thereof, including, e.g., a head portion, or an assembly of components which provide substantially the same functional activity. The portion may be a physical fragment of an intact phage, a selected product from normal or abnormal assembly of phage parts, or even an artificial or recombinant construct, e.g., from genetic manipulation of genes encoding (1) phage parts, (2) critical phage assembly components, or even (3) associated host genes which may be useful in ensuring phage replication or production. When referring to a phage genome, typically the term refers to a naturally occurring phage genome as set forth in SEQ ID NO: 63, but may include fragments, artificial constructs, mutagenized genomes including those found in AP50c, selected genomes, and “prophage” sequences, which are considered to be “defective” genomes which may have had segments deleted, inserted, or otherwise affected to disrupt normal genome function.

Typically, phage will be morphologically identifiable, having a size which is resolvable by imaging methods, e.g., electron microscopy. See, e.g., Ackermann and Nguyen (1983) Appl. Environ. Microbiol. 45:1049-1059.

An “AP50 phage” is a phage or phage-based construct (e.g., a phage tail, tail fragment, phage protein, or ghost phage) that inhibits the growth, survival, or replication of the target bacterium (e.g., B. anthracis). In some embodiments, the AP50 phage contains one or more mutations in its genome which enhance or increase lytic activity, including but not limited to, one or more nucleotide substitutions is at a position corresponding to nucleotide 271 of SEQ ID NO: 55 (i.e. nucleotide 271 of ORF 28) and/or a position corresponding to nucleotide 154 of SEQ ID NO: 63. In some embodiments, the AP50 phage is AP50c. Thus, an AP50 phage can include a portion of a phage that can be used to inhibit growth of the target bacterium. For example, an AP50 phage can be a portion of an intact phage that can be produced in a non-target bacteria. Thus, as defined herein, an AP50 phage can include a structural portion of an intact phage, e.g., a tail portion of a tailed phage; or an isolated protein component of an intact phage. These phage-based compositions include one or more proteins or protein domains derived from a natural or engineered bacteriophage. In some embodiments, the AP50 phage is unable to replicate, DNA or the phage itself, or assemble in a target bacterium, but nonetheless is capable of infecting the target bacterium so as to inhibit the growth, survival, or replication of the target bacterium.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed, or not expressed at all.

Certain embodiments of anti-bacterial phage include constructs which contain less than about 70, 50, 20, 5, 2, 1, 0.1 percent, or less of the parental phage nucleic acid content. The content may be either mass, or informational content, e.g., where some portion of the informational content is deleted.

As used herein, “target bacterium” or “target bacteria” refer to B. anthracis bacterium or bacteria whose growth, survival, or replication is inhibited by an AP50 phage. “Growth inhibition” can refer, e.g., to slowing of the rate of bacterial cell division, or cessation of bacterial cell division, and/or to death of the bacteria due to lysis by AP50 phage. In a typical embodiment, the “target bacterium” or “target bacteria” are pathogenic forms of B. anthracis. Examples of B. anthracis include, but are not limited to, the strains listed in Table 1 below and substrains thereof.

TABLE 1
Exemplary B. anthracis strains

| B. anthracis Strain | Source | Comments | AP50 Sens. | Gamma Sens. |
|---|---|---|---|---|
| ASC 004 | | Strain M36; used in vaccine | Yes | |
| | | research, U.K | | |
| ASC 006 | | Vollum 3b type strain. U.K. | Yes | |
| ASC 010 | | NCTC 2620. China. | Yes | |
| ASC 016 | | ATCC 937 | Yes | |
| ASC 025 | | U.K. bovine case presumed to be | Yes | Yes |
| | | caused by contaminated material | | |
| | | from Senegal. | | |
| ASC 027 | | U.K. bovine case presumed to be | Yes | Yes |
| | | caused by contaminated material | | |
| | | from Senegal. | | |
| ASC 031 | | U.K. bovine case presumed to be | Yes | Yes |
| | | caused by contaminated material | | |
| | | from Senegal. | | |
| ASC 032 | | Penicillin-resistant fatal human | Yes | Yes |
| | | case. U.K. | | |
| ASC 038 | | Fatal human case. U.K. | Yes | Yes |
| ASC 050 | | Zimbabwe (Human cutaneous | Yes | |
| | | isolate). | | |
| ASC 054 | | Zimbabwe (Human cutaneous | Yes | |
| | | isolate). Phage resistant. | | |
| ASC 061 | | Zebra. Etosha National Park. | Yes | |
| | | Namibia. | | |
| ASC 069 | | Human isolate. New Hampshire, | Yes | |
| | | U.S.A. | | |
| ASC 070 | | Penicillin resistant. | Yes | Yes |
| ASC 073 | | Zebra. Etosha N.P. Namibia. | Yes | Yes |
| ASC 074 | | Vulture feces, Etosha NP, Namibia. | Yes | |
| ASC 120 | | Australia. by MLVA | Yes | |
| ASC 131 | | Elephant skull. Zambia. | Yes | |
| ASC 152 | | Giraffe bone. Namibia. | Yes | Yes |
| ASC 158 | | Zebra. Etosha NP Namibia | Yes/No | No |
| ASC 159 | | Ames. Guinea pig re-isolate from | Yes | |
| | | vaccine challenge studies. U.K. | | |
| ASC 161 | | Ames. Guinea pig re-isolate from | Yes | Yes |
| | | vaccine challenge studies. U.K. | | |
| ASC 165 | | Ames. Guinea pig re-isolate from | Yes | Yes |
| | | vaccine challenge studies. U.K. | | |
| ASC 206 | | Kruger N.P. South Africa. | Yes | |
| ASC 254 | | Environmental isolate. U.K. | Yes | |
| | | Believed to be more than 100 years | | |
| | | old. | | |
| ASC 285 | | Environmental isolate. U.K. | Yes | Yes |
| | | Believed to be more than 100 years | | |
| | | old. | | |
| ASC 330 | | Ames re-isolate. U.K. | Yes | |
| ASC 386 | | Ames re-isolate with | Yes | |
| | | uncharacteristic colony morphology. | | |
| | | U.K. | | |
| ASC 394 | | Ames re-isolate from guinea pig | Yes | |
| | | which died despite ciprofloxacin | | |
| | | treatment. U.K. | | |
| ASC 398 | | Ames re-isolate from guinea pig | Yes | |
| | | which died despite doxycycline | | |
| | | treatment. U.K. | | |
| BDRD 01 | | Unknown A0089 strain | Yes | |
| A 0034 | | Bovine. China. | Yes | Yes |
| A 0039 | | Bovine. Australia. | Yes | Yes |
| A 0149 | | Human cutaneous isolate. Turkey. | Yes | Yes |
| A 0158 | | Bovine. Zambia. | Yes | Yes |
| A 0174 | | Canada | Yes | Yes |
| A 0188 | | Zebra. Etosha N.P. Namibia. | Yes | Yes |
| A 0248 | | Human. U.S. | Yes | Yes |
| A 0256 | | Human. Turkey. | Yes | |
| A 0264 | | Human. Turkey. | Yes | Yes |
| A 0267 | | Bovine. U.S.A. | Yes | Yes |
| A 0293 | | Sheep. Italy. | Yes | Yes |
| A 0328 | | Pig. Germany. | Yes | Yes |
| A 0376 | | Bovine. U.S.A. | Yes | |
| A 0379 | | Wool. Pakistan. | Yes | |
| A 0419 | | South Korea (fatal human case). | Yes | Yes |
| A 0442 | | Kudu, Kruger N.P. South Africa. | Yes | No |
| A 0462 | | Ames Guinea pig re-isolate from | Yes | |
| | | vaccine challenge studies (Porton | | |
| | | Down U.K.). | | |
| A 0463 | | Sheep. Pakistan. | Yes | No |
| A 0465 | | U.K. (Vollum). | Yes | |
| A 0489 | | Bovine. Argentina. | Yes | |
| ASC 008 | PCT | NCTC 109 (Paddington IV) | Yes | |
| ASC 009 | PCT | NCTC 1328 | Yes | Yes |
| ASC 018 | PCT | 958G | Yes | Yes |
| ASC 019 | PCT | 961G | Yes | |
| ASC 020 | PCT | 1012G | Yes | |
| ASC 023 | PCT | 1011 G | Yes | |
| ASC 024 | PCT | NP9 | Yes | |
| ASC 026 | PCT | A73/77 | Yes | Yes |
| ASC 028 | PCT | A187/78 | Yes | |
| ASC 030 | PCT | A191/78 | Yes | Yes |
| ASC 033 | PCT | C164G | Yes | Yes |
| ASC 035 | PCT | C11G | Yes | |
| ASC 036 | PCT | C129 G | Yes | Yes |
| ASC 040 | PCT | M84 | Yes | |
| ASC 042 | PCT | Denmark 79 | Yes | |
| ASC 046 | PCT | St2 | Yes | |
| ASC 063 | PCT | Etosha 86 | Yes | Yes |
| ASC 078 | PCT | Q78 | Yes | |
| ASC 080 | PCT | L9 (1) | Yes | Yes |
| ASC 091 | PCT | ATX 881017002 | Yes | Yes |
| ASC 127 | PCT | S6U1 | Yes | |
| ASC 149 | PCT | CT1264/07/88 | Yes | No |
| ASC 150 | PCT | AM1260/7/88 | Yes | |
| ASC 187 | PCT | F2909/90 | Yes | |
| ASC 193 | PCT | Landkey V13 | Yes | |
| ASC 209 | PCT | RNL 440 | Yes | |
| ASC 212 | PCT | RNL 443 | Yes | |
| ASC 214 | PCT | RNL 446 | Yes | |
| ASC 228 | PCT | Landkey 04 | Yes | |
| ASC 236 | PCT | Landkey R2I4 | Yes | |
| ASC 239 | PCT | E side North Kings Cross | Yes | |
| ASC 267 | PCT | Landkey sample 3 | Yes | Yes |
| ASC 278 | PCT | C300 | Yes | |
| ASC 279 | PCT | C313 | Yes | |
| ASC 296 | PCT | CO55 | Yes | |
| ASC 301 | PCT | CO61/93 | Yes | |
| ASC 306 | PCT | C317 | Yes | |
| ASC 308 | PCT | C323 | Yes | |
| ASC 309 | PCT | C325 | Yes | |
| ASC 310 | PCT | M8Y 040892 | Yes | |
| ASC 318 | PCT | DSM A74 | Yes | |
| ASC 336 | PCT | F | Yes | |
| ASC 338 | PCT | I | Yes | |
| ASC 339 | PCT | J | Yes | |
| ASC 340 | PCT | L | Yes | |
| ASC 354 | PCT | S10 | Yes | |
| ASC 362 | PCT | 93/37 | Yes | Yes |
| ASC 363 | PCT | 92/150 | Yes | Yes |
| ASC 369 | PCT | 92/123 | Yes | |
| ASC 373 | PCT | London 3 | Yes | |
| ASC 391 | PCT | AN 32/94 | Yes | |
| ASC 411 | PCT | 95/126 | Yes | Yes |

As used herein, “host bacterium” or “host bacteria” refer to a bacterium or bacteria used to produce, replicate, or amplify a phage, sometimes referred to as a parental phage, that is used to produce an anti-bacterial phage. Host bacteria or bacterium are also referred to as “host production bacterium” or “host production bacteria” throughout. One example of a host bacterium is B. anthracis Sterne strain 34F₂ (pXO1⁺ pXO2⁻). In one embodiment, the parental phage is a prophage, e.g., a defective or incomplete phage genome. Often the host production culture complements a defect in the phage, or suppresses a destructive function encoded in the phage. In other embodiments, the host production culture may make use of a helper phage to effect the capability.

AP50 phage can also include phage that comprise a mutation and cannot efficiently assemble into a replication competent phage in the target bacteria. Mutations can include mutations in genes that encode enzymes for replication of nucleic acids or genes that encode regulators of replication; or in genes that encode structural components of a phage or genes that encode regulators of the synthesis of structural components, or genes that encode proteins critical for assembly, e.g., assembly functions, or genes that regulate stoichiometry of proteins necessary for proper assembly. The mutations can be in the coding region of a gene or in a regulatory region of the gene, e.g., a promoter.

### Nucleic Acid Molecules

The present invention further provides nucleic acid molecules that encode any of the proteins having SEQ ID NO: 2, 4, 6, 8, 10, 12, 14, 16, 18, 20, 22, 24, 26, 28, 30, 32, 34, 36, 38, 40, 42, 44, 46, 48, 50, 52, 56, 58, 60, 62 (herein after referred to as a “phage protein”) and the related proteins herein described, preferably in isolated form. As used herein, “nucleic acid” is defined as RNA or DNA that encodes a protein or peptide as defined above, is complementary to a nucleic acid sequence encoding such peptides, hybridizes to any of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61 (herein referred to as a “phage nucleic acid” and ORF1, ORF2, ORF3, ORF4, ORF5, ORF6, ORF7, ORF8, ORF9, ORF10, ORF11, ORF12, ORF13, ORF14, ORF15, ORF16, ORF17, ORF18, ORF19, ORF20, ORF21, ORF22, ORF23, ORF24, ORF25, ORF26, ORF27, ORF28, ORF29, ORF30 and ORF31, respectively) across the open reading frame under appropriate stringency conditions, or encodes a polypeptide that shares at least about 85, 86, 87, 88, 89, 90, 91, 92, 93, 94, 95, 96, 97, 98, 99 or 100% sequence identity, with the entire contiguous amino acid sequence of any one of the phage proteins.

The “nucleic acids” of the invention further include nucleic acid molecules that share at least about 85, 86, 87, 88, 89, 90, 91, 92, 93, 94, 95, 96, 97, 98, 99 or 100% sequence identity with the nucleotide sequence of any of the phage nucleic acids, particularly across the open reading frame. Specifically contemplated are genomic DNA, cDNA, mRNA and antisense molecules, as well as nucleic acids based on alternative backbones or including alternative bases whether derived from natural sources or synthesized. Such nucleic acids, however, are defined further as being novel and unobvious over any prior art nucleic acid including that which encodes, hybridizes under appropriate stringency conditions, or is complementary to nucleic acid encoding a protein according to the present invention.

Homology or identity at the nucleotide or amino acid sequence level is deter mined by BLAST (Basic Local Alignment Search Tool) analysis using the algorithm employed by the programs blastp, blastn, blastx, tblastn and tblastx (Altschul et al. (1997) Nucleic Acids Res. 25, 3389-3402 and Karlin et al. (1990) Proc. Natl. Acad. Sci. USA 87, 2264-2268, both fully incorporated by reference) which are tailored for sequence similarity searching. The approach used by the BLAST program is to first consider similar segments, with and without gaps, between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (1994) Nature Genetics 6, 119-129 which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff et al. (1992) Proc. Natl. Acad. Sci. USA 89, 10915-10919, fully incorporated by reference), recommended for query sequences over 85 in length (nucleotide bases or amino acids).

For blastn, the scoring matrix is set by the ratios of N1 (i.e., the reward score for a pair of matching residues) to N (i.e., the penalty score for mismatching residues), wherein the default values for M and N are +5 and −4, respectively. Four blastn parameters were adjusted as follows: Q=10 (gap creation penalty); R=10 (gap extension penalty); wink=1 (generates word hits at every wine position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

“Stringent conditions” are those that (1) employ low ionic strength and high temperature for washing, for example, 0.015 M NaCl/0.0015 M sodium citrate/0.1% SDS at 50° C., or (2) employ during hybridization a denaturing agent such as formamide, for example, 50% (vol vol) formamide with 0.1% bovine serum albumin/0.1% Ficoll/0.1% polyvinylpyrrolidone/50 mM sodium phosphate buffer (pH 6.5) with 750 mM NaCl, 75 mM sodium citrate at 42° C. Another example is hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 0.1% SDS. A skilled artisan can readily determine and vary the stringency conditions appropriately to obtain a clear and detectable hybridization signal. Preferred molecules are those that hybridize under the above conditions to the complement of any of the phage nucleic acids and which encode a functional protein. Even more preferred hybridizing molecules are those that hybridize under the above conditions to the complement strand of the open reading frame of any of the phage nucleic acids.

As used herein, a nucleic acid molecule is said to be “isolated” when the nucleic acid molecule is substantially separated from contaminant nucleic acid molecules encoding other polypeptides.

The present invention further provides fragments of the encoding nucleic acid molecule. As used herein, a fragment of an encoding nucleic acid molecule refers to a small portion of the entire protein coding sequence. The size of the fragment will be determined by the intended use. For example, if the fragment is chosen so as to encode an active portion of the protein, the fragment will need to be large enough to encode the functional regions of the protein. For instance, fragments which encode peptides corresponding to predicted antigenic regions may be prepared. If the fragment is to be used as a nucleic acid probe or PCR primer, then the fragment length is chosen so as to obtain a relatively small number of false positives during probing/priming.

Fragments of the encoding nucleic acid molecules of the present invention (i.e., synthetic oligonucleotides) that are used as probes or specific primers for the polymerase chain reaction (PCR), or to synthesize gene sequences encoding proteins of the invention, can easily be synthesized by chemical techniques, for example, the phosphotriester method of Matteucci et al. (1981) J. Am. Chem. Soc. 103, 3185-3191 or using automated synthesis methods. Examples of such probes or primers include, but are not limited to, any of SEQ ID NO: 64 to 133. In addition, larger DNA segments can readily be prepared by well known methods, such as synthesis of a group of oligonucleotides that define various modular segments of the gene, followed by ligation of oligonucleotides to build the complete modified gene. In a preferred embodiment, the nucleic acid molecule of the present invention contains a contiguous open reading frame of at least about three-thousand and forty-five nucleotides.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides, and the like. A skilled artisan can readily employ any such label to obtain labeled variants of the nucleic acid molecules of the invention. Modifications to the primary structure itself by deletion, addition, or alteration of the amino acids incorporated into the protein sequence during translation can be made without destroying the activity of the protein. Such substitutions or other alterations result in proteins having an amino acid sequence encoded by a nucleic acid falling within the contemplated scope of the present invention.

The invention also encompasses oligonucleotides which hybridize to any region of a phage nucleic acid or the AP50 phage genome, including any of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, or 134. The invention encompasses synthetic oligonucleotides having chemical modifications from native nucleic acids, or nucleic acid constructs that express such anti-sense molecules as RNA. The oligonucleotide sequence can be complementary to the phage nucleic acids.

Oligonucleotides will generally be at least about 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. Typical oligonucleotides are usually not more than about 500, more usually not more than about 50, and even more usually not more than about 35 nucleotides in length, where the length is governed by efficiency of inhibition, specificity, including absence of cross-reactivity, and the like. It has been found that short oligonucleotides, of from seven to eight bases in length, can be strong and selective inhibitors of gene expression (see Wagner et al. (1996) Nat. Biotech. 14, 840-844).

Oligonucleotides may be chemically synthesized by methods known in the art (see Wagner et al. (1996) Nat. Biotech. 14, 840-844). Oligonucleotides of the invention can be chemically modified from the native phosphodiester structure, in order to increase their intracellular stability and binding affinity. A number of such modifications have been described in the literature, which alter the chemistry of the backbone, sugars, or heterocyclic bases.

### Recombinant DNA Containing a Phage Nucleic Acid

The present invention further provides recombinant DNA molecules (rDNAs) that contain a phage nucleic acid coding sequence. As used herein, a rDNA molecule is a DNA molecule that has been subjected to molecular manipulation in situ. Methods for generating rDNA molecules are well known in the art, for example, see Sambrook et al. (2005) Molecular Cloning—A Laboratory Manual, Cold Spring Harbor Laboratory Press. In the preferred rDNA molecules, a coding DNA sequence is operably linked to expression control sequences and/or vector sequences.

The choice of vector and/or expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as B. anthracis Sterne strain 34F₂ (pXO1⁺ pXO2⁻). A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention.

Any prokaryotic host can be used to express a rDNA molecule encoding a protein of the invention. The preferred prokaryotic host is E. coli.

Transformation of appropriate cell hosts with a rDNA molecule of the present invention is accomplished by well known methods that typically depend on the type of vector used and host system employed. With regard to transformation of prokaryotic host cells, electroporation and salt treatment methods are typically employed, see, for example, Sambrook et al. (2005) Molecular Cloning—A Laboratory Manual, Cold Spring Harbor Laboratory Press. With regard to transformation of vertebrate cells with vectors containing rDNAs, electroporation, cationic lipid or salt treatment methods are typically employed, see, for example, Graham et al. (1973) Virol. 52, 456; Wigler et al. (1979) Proc. Natl. Acad. Sci. USA 76, 1373-1376.

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern (1975) J. Mol. Biol. 98, 503-504 or Berent et al. (1985) Biotech. 3, 208-209 or the proteins produced from the cell assayed via an immunological method.

### Production of Recombinant Proteins

The present invention further provides methods for producing a phage protein of the invention using nucleic acid molecules herein described. In general terms, the production of a recombinant form of a phage protein typically involves the following steps:

A nucleic acid molecule is first obtained that encodes a phage protein of the invention, such as a nucleic acid molecule comprising, consisting essentially of or consisting of SEQ ED NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61. If the encoding sequence is uninterrupted by introns, as is this open reading frame, it is directly suitable for expression in any host.

The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

### The AP50 Phage Proteins

The present invention provides isolated proteins, allelic variants of the proteins, and conservative amino acid substitutions of the protein comprising the amino acid sequence of any of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60 and 62. As used herein, the “protein” or “polypeptide” refers, in part, to a protein that has the amino acid sequence depicted in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60 and 62. The terms also refer to naturally occurring allelic variants and proteins that have a slightly different amino acid sequence than that specifically recited above. Allelic variants, though possessing a slightly different amino acid sequence than those recited above, will still have the same or similar biological functions associated with these proteins. The methods used to identify and isolate other members of the family of proteins related to these proteins are described below.

The proteins of the present invention are preferably in isolated form. As used herein, a protein is said to be isolated when physical, mechanical or chemical methods are employed to remove the protein from cellular constituents that are normally associated with the protein. A skilled artisan can readily employ standard purification methods to obtain an isolated protein.

The proteins of the present invention further include insertion, deletion or conservative amino acid substitution variants of any of the phage proteins. As used herein, a conservative variant refers to alterations in the amino acid sequence that does not adversely affect the biological functions of the protein. A substitution, insertion or deletion is said to adversely affect the protein when the altered sequence prevents or disrupts a biological function associated with the protein. For example, the overall charge, structure or hydrophobic/hydrophilic properties of the protein can be altered without adversely affecting a biological activity. Accordingly, the amino acid sequence can be altered, for example to render the peptide more hydrophobic or hydrophilic, without adversely affecting the biological activities of the protein, In one example, ORF28 (SEQ ID NO: 56) has a single amino acid substitution of a isoleucine for leucine at amino acid 91.

Ordinarily, the allelic variants, the conservative substitution variants, and the members of the protein family, will have an amino acid sequence having at least about 85, 86, 87, 88, 89, 90, 91, 92, 93, 94, 95, 96, 97, 98, 99 or 100 amino acid sequence identity with the entire sequence set forth in any of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60 and 62. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. Fusion proteins, or N-terminal, C-terminal or internal extensions, deletions, or insertions into the peptide sequence shall not be construed as affecting homology.

Thus, the proteins of the present invention include molecules having the amino acid sequence disclosed in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60 and 62 and fragments thereof having a consecutive sequence of at least about 30, 31, 32, 33, 34, 35, 36, 37, 38, 39, 40, 41, 42, 43, 44, 45, 50, 55, 60, 65, 70, 75, 80, 85, 90, 95, 100, 105, 110, 115, 120, 125 or more amino acid residues of these proteins; amino acid sequence variants wherein one or more amino acid residues has been inserted N- or C-terminal to, or within, the disclosed coding sequence; and amino acid sequence variants of the disclosed sequence, or their fragments as defined above, that have been substituted by at least one residue. Such fragments, also referred to as peptides or polypeptides, may contain antigenic regions, functional regions of the protein identified as regions of the amino acid sequence which correspond to known protein domains, as well as regions of pronounced hydrophilicity. The regions are all easily identifiable by using commonly available protein sequence analysis software such as Mac Vector (Oxford Molecular).

Contemplated variants further include those containing predetermined mutations by, e.g., homologous recombination, site-directed or PCR mutagenesis, and the alleles or other naturally occurring variants of the family of proteins; and derivatives wherein the protein has been covalently modified by substitution, chemical, enzymatic, or other appropriate means with a moiety other than a naturally occurring amino acid (for example a detectable moiety such as an enzyme or radioisotope).

The present invention further provides compositions comprising a protein or polypeptide of the invention and a diluent. Suitable diluents can be aqueous or non-aqueous solvents or a combination thereof, and can comprise additional components, for example water-soluble salts or glycerol, that contribute to the stability, solubility, activity, and/or storage of the protein or polypeptide.

### Diagnostic Methods

The expression and activity of the AP50 phage may be used as a diagnostic marker for the identification of the presence of B. anthracis. For instance, a tissue sample may be assayed by any of the methods described above, and levels of lytic activity may be compared to the levels found in tissue which does not contain B. anthracis and/or does contain B. anthracis. Such methods may be used to diagnose or identify the presence of an infection by B. anthracis in a mammal, including a human.

In some embodiments, the present invention may be used to diagnose and/or monitor the treatment of B. anthracis infection with antibiotics. For example, at present a combination of several antibiotics is given to patients who have been exposed to B. anthracis. Tissue samples taken during treatment can be assayed for lytic activity to determine the presence and amount of B. anthracis present in the tissue sample. In some embodiments, the tissue sample is used to culture bacteria in the appropriate media, after which time the AP50 phage is added to the medium and lytic activity measured in the culture media. Suitable culture media include, but are not limited to, phage assay broth.

In one embodiment of the invention, cell cultures are grown from a sample suspected of containing B. anthracis and then subsequently tested for the presence of anthrax bacteria by the application of AP50 to cell cultures. Such a sample may be isolated from a swab. Cell culture isolates to be tested may be pure cultures or well-defined single colonies in a mixed bacterial population. If culture integrity with respect to age or purity is in doubt, the culture may be subcultured to produce isolated colonies on suitable culture media, such as e.g., 5% SBA. In one embodiment, suspect colonies selected for testing have following properties: nonhemolytic, opaque, slightly raised, irregular (although round colonies can form) with serrated edges, and gray-white with a ground-glass appearance. Suspect colonies typically show tenacity when the colony is probed with an inoculation loop or needle and disturbed. Spore suspensions with adequate concentration to yield confluent lawns may also be tested directly. Preferably, positive and negative control cultures are tested concomitantly. Inoculation of test samples and controls may be standardized via e.g., using a 1-μl loop, with which sufficient culture growth was removed to make an approximate 1-mm bead of cells, preferably from an individual colony. The growth is transferred to fresh plate such as e.g. afresh SBA plate by streaking a vertical line from the edge towards the center (approximately 1 in. in length) in the first quadrant.

A suitable amount of AP50 phage suspension (such as e.g. 5 μl) is placed on the agar surface. The location of the where the AP50 suspension is applied is noted. In one embodiment, after replacing the plate lid, circles are drawn on the lid above the sites where phage was applied. In the same embodiment, the sides of the plate lid and bottom are marked to allow for realignment of the top and bottom before the plates are read postincubation. The fresh cultures are then grown under suitable conditions. In one embodiment, the agar culture is incubated at 35° C.±2° C. for 20±4 hours. Preferably, the acceptance criteria for positive assay results are that there must be a clear zone (macroplaque approximately 5 to 10 mm in diameter) of no growth where phage was applied to the positive control in either the first or second quadrant. It is possible for a few colonies to emerge within the clear zone on the positive control, if such a control is used. A lawn of confluent growth must be present controls and test unknowns. A positive test yields plaque formation (which may be 5 to 10 mm in diameter) at the point of AP50 phage application after incubation. In one embodiment of the invention, positive test yields plaque formation 20±4 hours after incubation. Plaques may be seen in four to eight hours against the agar surface dulled by early bacterial growth around the site of AP50 phage application. To decrease the detection time and increase sensitivity, expression markers can be inserted into AP50 phage for earlier visual detection of lytic activity. In another embodiment of the invention, gamma phage is in combination with AP50 phage.

The method of the present invention will be used most frequently to screen for the presence of B. anthracis in a mixed population of bacteria derived from a biological sample as described herein. The mixed bacterial populations need not be selected prior to screening. Preparation of the sample prior to screening will generally not provide a homogeneous bacterial population, although it is possible to combine the screen of the present application with nutritional selection as described below.

In contrast to conventional phage transduction techniques intended to produce homogeneous colonies of transduced bacterial cells, the method of the present invention does not require that the transduced bacteria be isolated in any way. Instead, the screenable phenotype, e.g., a visually observable trait, conferred by the primary marker gene can be detected in a non-selected portion of the biological sample where viable, usually proliferating, non-target bacteria will be present. The screening can occur without selection since there is no need to isolate the transduced bacteria.

As described above, the assay of the present invention is useful for screening biological samples to determine whether B. anthracis present. The present invention is also useful for typing bacterial species and strains in a manner similar to conventional phage typing which instead relies on much slower plaque assays for determining phage infection.

For detection according to the present invention, AP50 phage is employed with or without gamma phage. The species and strain of the target B. anthracis may then be determined based on the pattern of lytic activity. Often, such tests may be run on a single carrier, where phage lysis are spotted in a fixed geometry or matrix on the carrier surface. Examples of such carriers include, but are not limited to, quantum dots. The pattern of reactivity may then be rapidly observed. In contrast to the previously-described screening methods, these typing methods will be useful in characterizing homogeneous bacterial cultures (i.e., contained on a single species or strain) as well as typing target bacteria in mixed populations.

In a specific embodiment, AP50 phage or plasmids encoding AP50 phage are modified to such that they contain or express a marker specific for bacterial cell lysis. The modified (or tagged) phage are introduced into, or mixed into, a sample environment in which they are to be followed. The sample environment can be any setting where bacteria exist, including outdoors (e.g., soil, air or water); on living hosts (e.g., plants, animals, insects); on equipment (e.g., manufacturing, processing or packaging equipment); and in clinical samples. The bacteriophage assay of the invention can then be carried out, using AP50 bacteriophage induced expression of the desired marker, and the presence of the tagged bacteria can be monitored or quantified. In one embodiment, the marker is a strepavidin-biotin system whereby expression of strepavidin by the AP50 phage results in binding to a carrier surface a subsequent detection at significantly lower level of lysis than is detectable by visual inspection. The use of such markers provides the advantage of decreasing assay time by detection of initial lytic activity which is not capable of being determined visually.

In another embodiment, RT-PCR is used to detect lytic activity. Oligonucleotides specific to a lytic marker are employed to detect lysis a levels below those that can be detected visually. In this embodiment, the marker may be either derived from the AP50 genome (e.g., any of ORF1-31) or may also be a gene exogenous to AP50 whose expression is linked to lytic activity. In this embodiment, detection time is also decreased by the increased sensitivity for detecting lysis by means other than visualization.

### Treatment Methods

The method for treating B. anthracis infections comprises treating the bacterial infection with a therapeutic agent comprising an effective amount of AP50 phage specific for the B. anthracis bacteria. The phage is administered in such a way as to directly induce lysis of the bacteria and/or express a lytic enzyme in an environment having a pH which allows for activity of said lytic enzyme. The AP50 phage can be used for the treatment or prevention of B. anthracis infection or also commonly known as anthrax.

A “bacterial infection” refers to growth of bacteria, e.g., in a subject or environment, such that the bacteria actually or potentially could cause disease or a symptom in the subject or environment. This may include prophylactic treatment of substances or materials, including organ donations, medical equipment such as a respirator or dialysis machine, or wounds, e.g., during or after surgery, e.g., to remove target bacteria which may cause problems upon further growth.

For example, if there is a B. anthracis bacterial infection of the upper respiratory tract, the infection can be prophylactically or therapeutically treated with a composition comprising an effective amount of at least one AP50 phage, and a carrier for delivering the phage to a mouth, throat, or nasal passage. It is preferred that the phage is in an environment having a pH which allows for lytic activity. If an individual has been exposed to someone with an infection of B. anthracis in the upper respiratory tract, the AP50 phage will reside in the mucosal lining and prevent any colonization of the B. anthracis infecting bacteria.

Infection of the B. anthracis bacteria by certain AP50 phage variants including, but not limited to AP50c, results in lysis of the bacteria. The therapeutic agent can contain one or more of these AP50 phage, and may also contain other phage capable of B. anthracis lysis including, but not limited to, gamma phage. The composition which may be used for the prophylactic and therapeutic treatment of B. anthracis infection includes the AP50 phage and a means of application (such as a carrier system or an oral delivery mode) to reach the mucosal lining of the oral and nasal cavity, such that the enzyme is put in the carrier system or oral delivery mode to reach the mucosa lining.

A “subject in need of treatment” is an animal with a bacterial infection that is potentially life-threatening or that impairs health or shortens the lifespan of the animal. The animal can be a fish, bird, or mammal. Exemplary mammals include humans, domesticated animals (e.g., cows, horses, sheep, pigs, dogs, and cats), and exhibition animals, e.g., in a zoo. In some embodiments, anti-bacterial phage are used to treat plants with bacterial infections, or to treat environmental occurrences of the target bacteria, such as in a hospital or commercial setting.

Prior to, or at the time the AP50 phage is put in the carrier system or oral delivery mode, it is preferred that the enzyme be in a stabilizing buffer environment for maintaining a pH range between about 4.0 and about 9.0, and more preferably between about 5.5 and about 7.5. The stabilizing buffer should allow for the optimum activity of the AP50 phage. The buffer may be a reducing reagent, such as dithiothreitol. The stabilizing buffer may also be or include a metal chelating reagent, such as ethylenediaminetetracetic acid disodium salt, or it may also contain a phosphate or citrate-phosphate buffer.

A “pharmaceutically acceptable” component is one that is suitable for use with humans, animals, and/or plants without undue adverse side effects (such as toxicity, irritation, and allergic response) commensurate with a reasonable benefit/risk ratio.

A “safe and effective amount” refers to a quantity of a component that is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a component effective to yield a desired therapeutic response, e.g., an amount effective to slow the rate of bacterial cell division, or to cause cessation of bacterial cell division, or to cause death or decrease rate of population growth of the bacteria. The specific safe and effective amount or therapeutically effective amount will vary with such factors as the particular condition being treated, the physical condition of the subject, the type of subject being treated, the duration of the treatment, the nature of concurrent therapy (if any), and the specific formulations employed and the structure of the compounds or its derivatives.

Means of application include, but are not limited to direct, indirect, carrier and special means or any combination of means. Direct application of the phage may be by nasal sprays, nasal drops, nasal ointments, nasal washes, nasal injections, nasal packings, bronchial sprays and inhalers, or indirectly through use of throat lozenges, or through use of mouthwashes or gargles, or through the use of ointments applied to the nasal nares, the bridge of the nose, or the face or any combination of these and similar methods of application. The forms in which the phage may be administered include but are not limited to lozenges, troches, candies, injectants, chewing gums, tablets, powders, sprays, liquids, ointments, and aerosols. The phage may also be placed in a nasal spray, wherein the nasal spray is the carrier.

The nasal spray can be a long acting or timed release spray, and can be manufactured by means well known in the art. An inhalant may also be used, so that the phage may reach further down into the bronchial tract, including into the lungs.

Any of the carriers for the AP50 phage may be manufactured by conventional means. However, it is preferred that any mouthwash or similar type products not contain alcohol to prevent deactivation and/or denaturation of the phage.

The phage may be added to these substances in a liquid form or in a lyophilized state, whereupon it will be solubilized when it meets body fluids such as saliva. The enzyme may also be in a micelle or liposome.

The effective dosage rates or amounts of the phage to treat the infection will depend in part on whether the lytic will be used therapeutically or prophylactically, the duration of exposure of the recipient to the infectious bacteria, the size, and weight of the individual, etc. The duration for use of the composition containing the enzyme also depends on whether the use is for prophylactic purposes, wherein the use may be hourly, daily or weekly, for a short time period, or whether the use will be for therapeutic purposes wherein a more intensive regimen of the use of the composition may be needed, such that usage may last for hours, days or weeks, and/or on a daily basis, or at timed intervals during the day. Any dosage form employed should provide for a minimum number of units for a minimum amount of time. The concentration of the active units of phage believed to provide for an effective amount or dosage of phage may be in the range of about 100 units/ml to about 100,000 units/ml of fluid in the wet or damp environment of the nasal and oral passages, and possibly in the range of about 100 units/ml to about 10,000 units/ml. More specifically, time exposure to the active phage units may influence the desired concentration of active enzyme units per ml. It should be noted that carriers that are classified as “long” or “slow” release carriers (such as, for example, certain nasal sprays or lozenges) could possess or provide a lower concentration of active (phage) units per ml, but over a longer period of time, whereas a “short” or “fast” release carrier (such as, for example, a gargle) could possess or provide a high concentration of active (phage) units per ml, but over a shorter period of time. The amount of active units per ml and the duration of time of exposure depends on the nature of infection, whether treatment is to be prophylactic or therapeutic, and other variables.

While this product and treatment may be used in any mammalian species such as farm animals including, but not limited to, horses, sheep, pigs, chicken, and cows, the preferred use of this product is for a human.

For the prophylactic and therapeutic treatment of anthrax, the AP50 phage may also be applied by direct, indirect, carriers and special means or any combination of means. Direct application of the phage may be by nasal sprays, nasal drops, nasal ointments, nasal washes, nasal injections, nasal packings, bronchial sprays and inhalers, or indirectly through use of throat lozenges, or through use of mouthwashes or gargles, or through the use of ointments applied to the nasal nares, the bridge of the nose, or the face or any combination of these and similar methods of application. The foi ins in which the phage may be administered include but are not limited to lozenges, troches, candies, injectants, chewing gums, tablets, powders, sprays, liquids, ointments, and aerosols. For the therapeutic treatment of anthrax, the bronchial sprays and aerosols are most beneficial, as these carriers, or means of distributing the composition, allow the phage to reach the bronchial tubes and the lungs.

The AP50 phage of the present invention can be administered via parenteral, subcutaneous, intravenous, intramuscular, intraperitoneal, transdermal, or buccal routes. For example, an agent may be administered locally to a site of injury via microinfusion. Alternatively, or concurrently, administration may be by the oral route. The dosage administered will be dependent upon the age, health, and weight of the recipient, kind of concurrent treatment, if any, frequency of treatment, and the nature of the effect desired.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### EXAMPLES

### Example 1

### Materials and Methods

Bacteria, phage and primers. B. anthracis and B. cereus sensu lato group strains were obtained from the Biological Defense Research Directorate collection (BDRD) and the phage AP50 was obtained from the Felix d′Herelle Reference Center for Bacterial Viruses, University of Laval, Quebec, Canada. Cells were grown in Luria-Bertani (LB) or phage assay (Nutrient broth 8 g/l, NaCl 5 g/l, MgSO₄ 0.2 g/l, MnSO₄ 0.05 g/l, CaCl₂ 0.15 g/l, pH adjusted to 5.9 with HCl) medium. B. anthracis Sterne strain 34F₂ (pXO1⁺ pXO2⁻) was used for propagation of AP50. A clear plaque mutant was picked and a pure line was obtained after 3 rounds of single plaque purification steps. B. thuringiensis strain HER1410 was used for propagation of phages Bam35c and Bth35646. Primers used in this study are provided in the sequence listing.

Preparation of Phage Stocks. Phage Stocks were Prepared by Confluent Lysis Method. Phages were collected from confluent plates by pouring 5 ml of phage assay broth on the plate and scraping the top agar. Agar particles and cell debris were removed by centrifugation (Beckman-Coulter Avanti J-20 XPI centrifuge, JA14 rotor, 8 K rpm, for 30 minutes at 4° C.) followed by filtration through a 0.45 μm filter. The resulting lysates were treated with DNase and RNase (1 for 1 hour at room temperature. The phage stocks were further concentrated by high speed centrifugation (Beckman-Coulter Avanti J-20 XPI centrifuge, JA20 rotor, 16, 000×g, for 2 hours at 4° C.) and the pellets were resuspended in 1/10^{th} volume of PBS or PA broth. The titer of the stocks were determined on 34F₂ (plates were incubated overnight at 25° C.) and the stocks were stored at 4° C.
Determination of burst size. B. anthracis spores (1×10⁷) were germinated by growing in 1 ml of phage assay broth at 37° C. shaker for 1 hour and infected with AP50 at a multiplicity of one. The phages were allowed to adsorb to the cells without shaking at 37° C. or at room temperature for 30 minutes or 45 minutes, respectively. The cell-phage mixture was serially diluted and plated with indicator bacteria (34F₂) to determine the infective centers (ICs). The dilutions were further incubated for 2 hrs and aliquots were taken at different time points and plated to enumerate plaque forming units (PFU). The burst size was calculated by dividing the PFU after 2 hours of incubation by the initial IC.
Scanning electron microscopy. Wild type B. anthracis Sterne strain 34F₂ and an AP50^{R} mutant derivative were infected with AP50c phage at a multiplicity of one and incubated at room temperature for 45 minutes, followed by the addition of 2.5% EM grade glutaraldehyde (Ted Pella, Inc) to fix the cells. SEM was performed at Dennis Kunkel microscopy, Inc.
Isolation of phage DNA. Phage lysate (1×10¹⁰ pfu/ml) in PBS was treated with proteinase K (266 μg/ml) and RNase (26.6 μg/ml) for 30 mM at 37° C. followed by incubation for 30 minutes at 56° C. Phage particles were disrupted by adding SDS and EDTA to final concentrations of 1% and 0.05 M, respectively, and incubating the mixture for 5 minutes at room temperature. The solution was extracted with phenol, phenol:chloroform:isoamylalcohol and chloroform:isoamylalcohol and the DNA was precipitated by adding sodium acetate (final concentration of 0.3M) and 2.5 volumes of ethanol. The precipitated DNA was pelleted by centrifuging in a microcentrifuge at 16,000 g for 30 minutes at 4° C. followed by a wash with 70% ice cold ethanol. The final pellet was air dried and resuspended in TE (10 mM Tris-HCl pH 8.0, 1 mM EDTA).
DNA Sequencing of phage genome. AP50 genome sequence was determined by pyrosequencing method in GS20 sequencer (Roche/454 Life Sciences). The workflow of the GS20 system involved generation of a single-stranded template DNA library, emulsion-based clonal amplification of the library by emPCR, data generation via sequencing-by-synthesis followed by data analysis using different bioinformatic tools. The library consisted of a set of random fragments that represented the entire genome. These random fragments were generated by nebulizing 5 μg of starting DNA to an average size between 300 to 800 nucleotides.

Short adaptors (A and B), specific for both the 3′ and 5′ ends, were added to each fragment. The adaptors were used for purification, amplification, and sequencing steps. Single-stranded fragments with A and B adaptors composed the sample library used for subsequent workflow steps. The single-stranded DNA library was immobilized onto specifically designed DNA capture beads. The bead-bound library was emulsified with amplification reagents in a water-in-oil mixture. Each unique sample library fragment was amplified within its own microreactor. The clonally amplified fragments were enriched and loaded onto a PicoTiter Plate device for sequencing. Addition of one (or more) nucleotide(s) complementary to the template strand results in a chemiluminescent signal recorded by the CCD camera of the Genome Sequencer Instrument. The combination of signal intensity and positional information generated across the PicoTiter Plate device allows the software to determine the sequence of more than 400,000 individual reads per 7.5-hour instrument run simultaneously. The resulting sequence data were assembled de novo using 454 Life Sciences Newbler® software.

Computational analyses. Preliminary identification of the open reading frames (ORFs) of AP50 genome was done using Vector NTIE (Invitrogen) software. Gene assignments were made if ribosome binding sites upstream of the putative ORFs (close match to the sequence AGGAGG) were present. Further, AP50 ORFs were aligned with the annotated ORFs of the genomes of other Gram-positive tectiviral phages: Bam35 (GenBank Accession No. AY257527), Gil16 (AY701338) and pBC1 in15 (AE016878). Protein alignments were done using the identity matrix Blossum62. Possible homologies to known proteins were searched with PSI-BLAST. The solubility and domain prediction for each putative gene product was done with SMART web interface.
Identification of the mutations in clear and turbid plaque variants. To identify the mutations in clear and turbid plaque variants of AP50, a single plaque was suspended in one ml of water, filtered through 0.45 μm filter and 1 μl of this lysate was used as template in PCR using the primers of SEQ ID NO: 64 to 133. The resulting PCR fragments were sequenced in an ABI 3730 sequencer using the PCR and additional internal primers.

### Example 2

### Comparative Analysis Between of Bacillus Species to Lysis by Modified AP50

To determine the specificity of modified AP50 a comparative analysis was conducted. Table 1 shows the results of a side by side comparative analysis between AP50 and γ phage in B. anthracis. As shown in Table 2, approximately 4.9% of B. anthracis colonies were resistant to lysis by modified AP50 while 12.2% of B. anthracis colonies were resistant to lysis by gamma phage. Therefore, the modified AP50 exhibits equivalent or better lytic potential against B. anthracis than gamma phage.

TABLE 2
Comparative analysis between AP50 modified and Gamma phage

| | Phage | | | |
|---|---|---|---|---|
| | AP50 (modified) | | Gamma phage | |
| B. anthracis | 39/41 | 2/41 | 36/41 | 5/41 |

Table 3 shows the results of a comparative analysis of lysis in various Bacillus species after infection by the AP50 modified phage. As illustrated in Table 3, all B. cereus sensu lato were resistant to lysis by modified AP50 compared to 90% for gamma phage. Therefore, the inventive modified AP50 is potentially more specific than gamma phage.

TABLE 3
Comparative analysis of lysis in various Bacillus
species after infection by the AP50 modified phage

AP 50 (modified)

| Bacteria | Sensitive | Resistant |
|---|---|---|
| B. anthracis | 103/112 | 9/112 |
| B. cereus sensu lato | 0/100 | 100/100 |

### Example 3

### Stability of AP50c

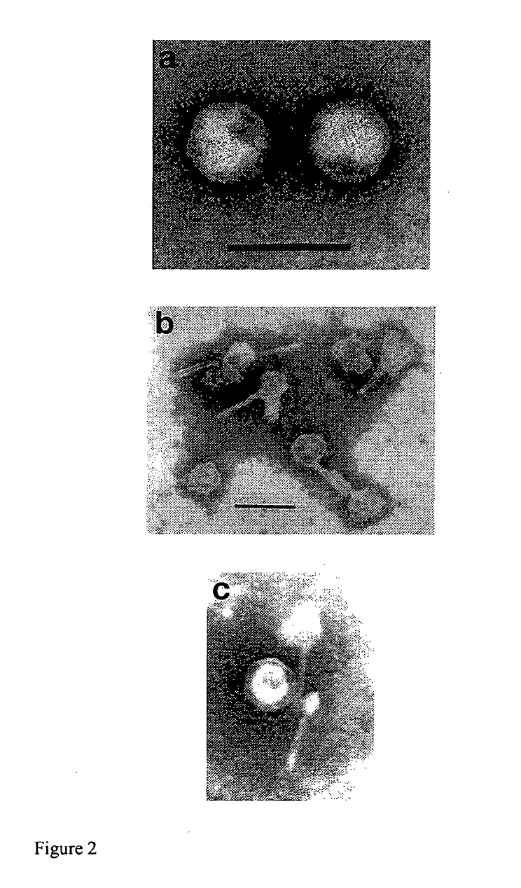

As seen with other tectiviral phages, AP50c is highly sensitive to chloroform treatment losing viability rapidly. Treatment with 1% chloroform reduced the viability to less than <10⁻⁸ in 1 hour at 37° C. Electron microscopic examination of chloroform treated phage particles showed collapsed empty viral heads and a pseudotail (see FIG. 2A, 2C). AP50c requires divalent cations for stability since phage particles were found to be more stable in phage assay medium containing Ca⁺⁺, Mg⁺⁺ and Mn⁺⁺ than in phosphate buffered saline (see Table 4 below). Incubation of phage particles in PBS at 37° C. overnight reduced the viability three orders of magnitude compared to incubation in phage assay broth. A similar trend was seen on long term storage at room temperature. In general, AP50c was found to be more stable in phage assay broth at 4° C.

TABLE 4
Stability of modified AP50 under various conditions

| Condition | Efficiency of Plating^{b} |
|---|---|
| Phage assay (PA medium) | 2 × 10⁻¹ |
| Phosphate buffered saline (PBS) | 1 × 10⁻³ |
| PBS + PA broth salts | 9 × 10⁻² |
| Chloroform | 2 × 10⁻⁸ |

^{a}overnight incubation at 37° C.
^{b}the ratio of titer on the condition examined over untreated
^{c}1 hour at 37° C.

While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, and patent applications referred to in this application are herein incorporated by reference in their entirety.

1. An isolated Bacillus phage AP50 containing one or more nucleotide substitutions in the phage genome, wherein the one or more nucleotide substitutions increase lytic activity of the phage.

2. The isolated Bacillus phage AP50 of claim 1, wherein the one or more nucleotide substitutions is at a position corresponding to nucleotide 271 of SEQ ID NO: 55.

3. The isolated Bacillus phage AP50 of claim 2, wherein the substitution at nucleotide 271 is a C to T substitution.

4. The isolated Bacillus phage AP50 of claim 1, wherein the substitution is at a position corresponding to nucleotide 154 of SEQ ID NO: 63.

5. The isolated Bacillus phage AP50 of claim 4, wherein the substitution at nucleotide 154 is a T to C substitution.

6. The isolated Bacillus phage AP50 of claim 1, wherein the phage genome comprises the nucleotide sequence of one or more of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61 or the complement thereof.

7. The isolated Bacillus phage AP50 of claim 4, wherein the phage genome comprises the nucleotide sequence of SEQ ID NO: 63 (full length genome) or the compliment thereof.

8. A composition comprising the isolated bacteriophage of claim 1.

9. The composition of claim 8, wherein the composition further comprises gamma phage.

10. A kit comprising the composition of claim 8.

11. An isolated nucleic acid selected from the group consisting of:
(a) an isolated nucleic acid encoding a protein comprising the amino acid sequence of any of SEQ ID NO: 2, 4, 6, 8, 10, 12, 14, 16, 18, 20, 22, 24, 26, 28, 30, 32, 34, 36, 38, 40, 42, 44, 46, 48, 50, 52, 56, 58, 60, or 62; and
(b) an isolated nucleic acid comprising any of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, or 61, and
(c) an isolated nucleic acid with at least 85% sequence identity to any of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, or 61.

12. An isolated nucleic acid comprising SEQ ID NO: 63.

13. A recombinant phage comprising the nucleic acid of claim 11.

14. A recombinant phage comprising the nucleic acid of claim 12.

15. An isolated protein selected from the group consisting of:
(a) an isolated protein comprising the amino acid sequence of any of 2, 4, 6, 8, 10, 12, 14, 16, 18, 20, 22, 24, 26, 28, 30, 32, 34, 36, 38, 40, 42, 44, 46, 48, 50, 52, 56, 58, 60, or 62, and
(b) an isolated protein with at least 85% sequence identity to any of SEQ ID NO: 2, 4, 6, 8, 10, 12, 14, 16, 18, 20, 22, 24, 26, 28, 30, 32, 34, 36, 38, 40, 42, 44, 46, 48, 50, 52, 56, 58, 60, or 62.

16. A method for detecting the presence of B. anthracis in a subject comprising:
(a) isolating a biological sample from the subject,
(b) contacting a sample with the phage of claim 1, and
(c) detecting for the presence of bacterial lysis, wherein the increased presence of bacterial lysis compared to a control indicates the presence of B. anthracis in the sample.

17. The method of claim 16, wherein (a) further comprises incubating said biological sample under conditions sufficient to induce growth of B. anthracis.

18. The method of claim 16, wherein said control is a sample which does not contain B. anthracis.

19. The method of claim 16, wherein the contacting in (b) is carried out under conditions sufficient to induce phage lysis of B. anthracis.

20. The method of claim 16, further comprising contacting the biological sample with gamma phage.


SEQUENCE LISTING (134 sequences)

SEQ ID NO: 1 - DNA, 183 bp, Artificial
  OFR1
atgctattctacactgtaaaggaatttgcggaaatggctaagatttcagagaagactgtgactaggtacatcaagacgggtgatttggaagcagtcaagtttggcggacaatggcgtatcactgaaacagcggtacaaagctatattaaaaataattcaaatatcggaggacgagaaaatgac

SEQ ID NO: 2 - protein, 61 aa, Artificial
  Protein encoded by ORF1
MLFYTVKEFAEMAKISEKTVTRYIKTGDLEAVKFGGQWRITETAVQSYIKNNSNIGGREND

SEQ ID NO: 3 - DNA, 540 bp, Artificial
  ORF2
atgactaataaaaatgaacaattaaataatggtgctgtaacatcttatgtggatactgacatgcttgctaatggggaacaaccaatggtaattactcctgaagtaaatacaaatgacatcgtgacagtggacaaggtgtcaccatctattttcgaagctcaaggcggtaaggatgtattcttttcttctattcaaacgaaagaccgtaaatcagctatcaaagtctacaacgctatcaattctagcgaaaatcctttagcagatcatcgtggtgaagtgttacatatcactgacatggttgcacatgcaattacattagaagatgatgttacaaaagaagatgtggacgcattacgtgtcgtattagtggacaaggacggtaaagcataccatgcaatttcacaaggtgttgtgtcatctattcaaaagattattagcatcgttggaccagcaccatggacagacgagccacttgaaatcgtacctgtagaagtgaagacacgtaaaggattcaaaacattaactctacaattacaaggt

SEQ ID NO: 4 - protein, 180 aa, Artificial
  Protein encoded by ORF2
MTNKNEQLNNGAVTSYVDTDMLANGEQPMVITPEVNTNDIVTVDKVSPSIFEAQGGKDVFFSSIQTKDRKSAIKVYNAINSSENPLADHRGEVLHITDMVAHAITLEDDVTKEDVDALRVVLVDKDGKAYHAISQGVVSSIQKIISIVGPAPWTDEPLEIVPVEVKTRKGFKTLTLQLQG

SEQ ID NO: 5 - DNA, 222 bp, Artificial
  ORF3
gtggatggtataaaagaagtggtacaagtttctattaaaaccaatgagcgtgacatcgaattcacatcatttgcaggattgaatcaaattaaacaatcgcttgatggtgatgtgtcatcggtcgtattaacggatgaagaactagaaatgatgttatgtgtgaagcagcgtgataagttagcatcattcttccaatcggtgctagaacgtaagaatgcggta

SEQ ID NO: 6 - protein, 74 aa, Artificial
  Protein encoded by ORF3
VDGIKEVVQVSIKTNERDIEFTSFAGLNQIKQSLDGDVSSVVLTDEELEMMLCVKQRDKLASFFQSVLERKNAV

SEQ ID NO: 7 - DNA, 699 bp, Artificial
  ORF4
atggctaataaacgtcaacgtaaaaagatagtgaaaaagaaacaagaatcctttttatcatcggtgggttattcgaagaaacaaatgaaaacgattagcacaactgatagagcaaaggtagtaaagaaagaaacatataaaaagaaaaagcgtgacaagtatcaccaagcaagatcaatggggtttggttctaaagaagcaaacaaaatgagtagttggtctgactcacgctttataaagtatatagaagaatttaattcttactatatgattgtcatgtataaagatgtaacagaagagacggacagcgaagcattacatatgattaagaaccacacgaaaagacgtagcacatccaacttacttagaagtattaaaggatggttgtctgtagataaaaatcaaggttatataggtggatatgaaattcaagtaggtaagaaagacgaaatagatttccatttatatgcttataaacagcgaaagtatttacaagcatatagaggacaagggttacaattaaaacctctattaaacttattagaaaatatgatggtattattatacatggtagaagctaaagaccaatttgttgaggacttatgtacaaacttgagaaagctaccatatgaacaggcacatataaacgctaattatatagaagaagaatttataacagatagaagtgacttgcatttt

SEQ ID NO: 8 - protein, 233 aa, Artificial
  Protein encoded by ORF4
MANKRQRKKIVKKKQESFLSSVGYSKKQMKTISTTDRAKVVKKETYKKKKRDKYHQARSMGFGSKEANKMSSWSDSRFIKYIEEFNSYYMIVMYKDVTEETDSEALHMIKNHTKRRSTSNLLRSIKGWLSVDKNQGYIGGYEIQVGKKDEIDFHLYAYKQRKYLQAYRGQGLQLKPLLNLLENMMVLLYMVEAKDQFVEDLCTNLRKLPYEQAHINANYIEEEFITDRSDLHF

SEQ ID NO: 9 - DNA, 2194 bp, Artificial
  ORF5
gtgagtaataaacaaaaaaaagagcgtcaaaagcctgcgaagcttttaacgctggacacggaaacacgaggtttgacgggcaacgtgtttcgtgtcggattgtttgacggtacaaattactataaatcaaatacctttgacgagattcttgatttatttgaacagtataaagattatgagtgtcacgtatacgtccataatttagatttcgatttagctaaaattgcaactactctatttaaacgtgatagggtgcggttcgctaaatccatctttattaatggtaacgttgtgacattacattctgactctatgatactacatgatagccttagattgctacctggaagtttagaaaaattatgtaaggatttcggattaaccgacaatgcaaagaaagatttgtcggaagttatcaaagaacagggatatgcggtatataaaaaagatggtgtgacgtttgataaaaagaaatcgttaggtaactattttgaaaacgtaccagctgacgatccaacgctaaatgagtatttagaatttgactgtcgctcactgtatgaaatattaacaattgttatggacatagctaatataggtcttgaaacacttgtgatgtgtccaacaacagcttctcttgctatgagagtgtataaggaacaatatcgtgaacagtatgataaagtagcaacacatttttatatgggtgaatggggacaatttttagaagagcatgtacgacaatcctattatggaggtcgtacagaagtatttacaccacacttaccacatggttatcactatgacgtaaacagtttatatccgtatgtcatgaaaattgcaaagtttcctgtaggctatccaaaccttttaaaagatggacaagccgcgacgaaatggaaacactggaaacgtagagcaataggtgggggtgttatgtggtgtcgtgtagatgtacccgaggatatgtatatacctgtattacctaaacgtgacccgagcgggaaactattattccctgtaggtaagctagaaggtgtatggacattaccagagttattagaagctgaaaagaatggttgcacgatagaagcaatctatcaaatggtatattgggaacacatggaaccgatattcaaagagtttgttgagcattttgaggacttgaaaaagaactctaaaggagcaaaacgaacatttgcaaaacttattcagaatagcttgtatgggaagtttggtatgaatagggtgcgtgtcagtttgggggacatggaagaccgctatgatctacacgaaaagcaaataccatataaagaatttaaacatgattgtaatgggttaacactagaatttattcaatatataagcgaaagtaaggctagttacatacaacctcatattgcaacctatgtgacagcctatgcacgtatccttttatttagagggttaaaggaacaagctagtaaaggtgtgctaggatactgtgacacggattctattgcaggtacggcaaaaatgcctgatgaaatgattcatgatgaagattatggtaagtgggcgttagaaggtgaactagaagaaggtatattcttgcaacccaaattctatgcggaacgctatacaaacggaaaagaagtcattaaggcaaaaggaatacctcgtgagaaaatggaagagttgtcttttgagaattacaaggaatggcttgaaatcatgaaagaagggcaacaggaacgtatcgacattttcgaaggatatgagtcacgcaagaaattttcaacaacattaaaagcatctgaggattttgacacattacgtgaaatgaagaaatctattaatttattattagaacaaaagcgtgacattgattataaagggaatgtaacaagacctcataaacgttacgattacggggataagaaggacaagattgattatgaagattataagtctagagaagataaattaaacaatatgtatgatgatgtagacgatctaaaagagcaagtggacgaaataggttacatcaaatgtatgaaacaaggtgacatgtattttgaggaatacaaacatttaacaaagtcagttaaaagtaaatattttcgtagaacggggacacctatagacgtatgggcgaatgagtcgggatgggatgttaacgaattactagaagaattacgattgatgggggtatgtt

SEQ ID NO: 10 - protein, 731 aa, Artificial
  Protein encoded by ORF5
VSNKQKKERQKPAKLLTLDTETRGLTGNVFRVGLFDGTNYYKSNTFDEILDLFEQYKDYECHVYVHNLDFDLAKIATTLFKRDRVRFAKSIFINGNVVTLHSDSMILHDSLRLLPGSLEKLCKDFGLTDNAKKDLSEVIKEQGYAVYKKDGVTFDKKKSLGNYFENVPADDPTLNEYLEFDCRSLYEILTIVMDIANIGLETLVMCPTTASLAMRVYKEQYREQYDKVATHFYMGEWGQFLEEHVRQSYYGGRTEVFTPHLPHGYHYDVNSLYPYVMKIAKFPVGYPNLLKDGQAATKWKHWKRRAIGGGVMWCRVDVPEDMYIPVLPKRDPSGKLLFPVGKLEGVWTLPELLEAEKNGCTIEAIYQMVYWEHMEPIFKEFVEHFEDLKKNSKGAKRTFAKLIQNSLYGKFGMNRVRVSLGDMEDRYDLHEKQIPYKEFKHDCNGLTLEFIQYISESKASYIQPHIATYVTAYARILLFRGLKEQASKGVLGYCDTDSIAGTAKMPDEMIHDEDYGKWALEGELEEGIFLQPKFYAERYTNGKEVIKAKGIPREKMEELSFENYKEWLEIMKEGQQERIDIFEGYESRKKFSTTLKASEDFDTLREMKKSINLLLEQKRDIDYKGNVTRPHKRYDYGDKKDKIDYEDYKSREDKLNNMYDDVDDLKEQVDEIGYIKCMKQGDMYFEEYKHLTKSVKSKYFRRTGTPIDVWANESGWDVNELLEELRLMGVC

SEQ ID NO: 11 - DNA, 201 bp, Artificial
  ORF6
atgttaaccgatagagaacaagaagcgttagcgtgtataagcgggtatatgagacaaaacgggtttgcaccatccgtgcgagaaatggctggtttattatttgtcagtcacaagaccgcacatcgttatatgattcaattagaaaccaaaggacacattaagagaatacatcatcgttcacgtgctatccaactatgtgta

SEQ ID NO: 12 - protein, 67 aa, Artificial
  Protein encoded by ORF6
MLTDREQEALACISGYMRQNGFAPSVREMAGLLFVSHKTAHRYMIQLETKGHIKRIHHRSRAIQLCV

SEQ ID NO: 13 - DNA, 135 bp, Artificial
  ORF7
atgcgtgataaagttttagacttaatcattgaactatccaaatcaacaaaacaggtcgtagcaaaagatttcattattaatgaattatataaaatagcaaaagaagatgaatcgaaagagaaggagactagcaaa

SEQ ID NO: 14 - protein, 45 aa, Artificial
  Protein encoded by ORF7
MRDKVLDLIIELSKSTKQVVAKDFIINELYKIAKEDESKEKETSK

SEQ ID NO: 15 - DNA, 87 bp, Artificial
  ORF8
atgctagtctttttatttgtccaaaatgacacatttgtgacgttacaagcgaacatatgtttgggttatacttgtcacatcaagcaa

SEQ ID NO: 16 - protein, 29 aa, Artificial
  Protein encoded by ORF8
MLVFLFVQNDTFVTLQANICLGYTCHIKQ

SEQ ID NO: 17 - DNA, 231 bp, Artificial
  ORF9
atggaaggtattgatttaagtcatgtgcaatgttcattgccacctattccaaacccgttaacgtttgaagatttaacagaagaacaatttaaagcgttattaaatgttattcatgattttaaattcgcttgtagagaaagtaatttaccacttgctttctattatgtattagaaaaatgtactgaccctgtagtgaaacaagaattaatagatgctcatagatatgggtgt

SEQ ID NO: 18 - protein, 77 aa, Artificial
  Protein encoded by ORF9
MEGIDLSHVQCSLPPIPNPLTFEDLTEEQFKALLNVIHDFKFACRESNLPLAFYYVLEKCTDPVVKQELIDAHRYGC

SEQ ID NO: 19 - DNA, 171 bp, Artificial
  ORF10
atgttcaaaacattatcaaaactgtaccgtgacttattacatcaaaacatagatttgcacaatgaaaatacaaaactacgattacaaaatgcacggctgcaaagtaagttagcaaccgctgaaatagatttataccattttaaaaattcaatagaaaggatgattaacaaa

SEQ ID NO: 20 - protein, 57 aa, Artificial
  Protein encoded by ORF10
MFKTLSKLYRDLLHQNIDLHNENTKLRLQNARLQSKLATAEIDLYHFKNSIERMINK

SEQ ID NO: 21 - DNA, 342 bp, Artificial
  ORF11
atgacagactcattacaagtagtggaagaaaaaacgaaatttagattaggagattttcaattctttgcaaagaagaaagaagaggacgatcaagaggaagaagaggaacttgaggaagaggaagaagaggaagaagaaaagccgaaaccaaaacgtaaatcaaaaagcggggaggatgcaccagcatgggcacaagaaataatcggactgttgaaaccgaaagcggaggaacagcaaacaaaacagaaagtaccagtacccgaagcaccaatagtggaggacgaggaagaggaagaaccgccgaaagccagtccactgaaaagcttcctaagtcggttgtgg

SEQ ID NO: 22 - protein, 114 aa, Artificial
  Protein encoded by ORF11
MTDSLQVVEEKTKFRLGDFQFFAKKKEEDDQEEEEELEEEEEEEEEKPKPKRKSKSGEDAPAWAQEIIGLLKPKAEEQQTKQKVPVPEAPIVEDEEEEEPPKASPLKSFLSRLW

SEQ ID NO: 23 - DNA, 705 bp, Artificial
  ORF12
atgggcacaagaaataatcggactgttgaaaccgaaagcggaggaacagcaaacaaaacagaaagtaccagtacccgaagcaccaatagtggaggacgaggaagaggaagaaccgccgaaagccagtccactgaaaagcttcctaagtcggttgtggtagaagtcccgaacggtgagaagtcacccgaggacaccaaaaaggaagaacaacgaaaggcagcagccgcacgaaaacgtaaatcacgtgcagcagccgcaacgaaaaagaagtcgtcaccatctattggtgatgcaacgcaattaaaagtattgcttcttactacgtcacagatcatagcagcaagagaaggtatgagcgtatgggcgatgacggaacaagaggttgaccaaattgttacaccgctttatagcatcctatctaaaaatgatggggtggggcaagtcatgggtgaatatgccgaccacattgctttaatcgtggcagcatttactatatttgtaccaaaatttatgatgtggaaagcatcaagacctaagaaggagggaacgcactatgctagaccaaatccaaattccaaacgagaacaaggaaagcaaacaggagaggttgcaactagtagtagaccaagtggtggacagcctaccaacaacggtacgacttttggcgggcagctatctgaactcgttccgccaagtgctggaatc

SEQ ID NO: 24 - protein, 235 aa, Artificial
  Protein encoded by ORF12
MGTRNNRTVETESGGTANKTESTSTRSTNSGGRGRGRTAESQSTEKLPKSVVVEVPNGEKSPEDTKKEEQRKAAAARKRKSRAAAATKKKSSPSIGDATQLKVLLLTTSQIIAAREGMSVWAMTEQEVDQIVTPLYSILSKNDGVGQVMGEYADHIALIVAAFTIFVPKFMMWKASRPKKEGTHYARPNPNSKREQGKQTGEVATSSRPSGGQPTNNGTTFGGQLSELVPPSAGI

SEQ ID NO: 25 - DNA, 249 bp, Artificial
  ORF13
atgctagaccaaatccaaattccaaacgagaacaaggaaagcaaacaggagaggttgcaactagtagtagaccaagtggtggacagcctaccaacaacggtacgacttttggcgggcagctatctgaactcgttccgccaagtgctggaatctgaacagcatgacattgacggaaacattgatttagccttgtcacgtttacgtgagtacatcgactatatccaatatggtcacgatcaagaaaatgag

SEQ ID NO: 26 - protein, 83 aa, Artificial
  Protein encoded by ORF13
MLDQIQIPNENKESKQERLQLVVDQVVDSLPTTVRLLAGSYLNSFRQVLESEQHDIDGNIDLALSRLREYIDYIQYGHDQENE

SEQ ID NO: 27 - DNA, 636 bp, Artificial
  ORF14
atggaacgtatcccaaaagaccaacacgtatttatcacgggacaaacgggtacggggaaatcttttcttgctgaaacgtatttagcaggttacgaacatgtaattaagttagatacaaaaggtgaggtgtttgaaagacgaaaaaagaaacagcctgtatggcgtgggttacgtgaaggaaaggactttacagtcatagagcatttagacgaaatcgacagcgtggaaacaaagaaaatcatttatgcacctgtctttcaagagcaagaaatggaatactatgatgctctcatgcaatgggtgtacaggagagaaaatacacaattatggattgatgaactcatggaagtatgcccgagtcctttcaaataccctccctacctcaaaggtcttatgactagaggacgttcaaaagaagctactgtgtgggcgtgtacgcaacgcccgagtgacattccttctattgtaatggggaatagtgaccactttttcgtctttgaccaaaacttgccacaagatcgtaagaagttatgtgagacaacgggtagttataagtttatggaattaccgggctatcgtaacttttggtattttaagcgtggcatgacagatcccgtactcgccacattgaaatta

SEQ ID NO: 28 - protein, 212 aa, Artificial
  Protein encoded by ORF14
MERIPKDQHVFITGQTGTGKSFLAETYLAGYEHVIKLDTKGEVFERRKKKQPVWRGLREGKDFTVIEHLDEIDSVETKKIIYAPVFQEQEMEYYDALMQWVYRRENTQLWIDELMEVCPSPFKYPPYLKGLMTRGRSKEATVWACTQRPSDIPSIVMGNSDHFFVFDQNLPQDRKKLCETTGSYKFMELPGYRNFWYFKRGMTDPVLATLKL

SEQ ID NO: 29 - DNA, 138 bp, Artificial
  ORF15
gtggaggggaaatttgcagggattggactgaaaaatattttagccatcttctttttattcattgtatttatagttgttgctaaagtaattttcacgaagtatcccataaaaggcgttagtgaagtaatacaaactgta

SEQ ID NO: 30 - protein, 46 aa, Artificial
  Protein encoded by ORF15
VEGKFAGIGLKNILAIFFLFIVFIVVAKVIFTKYPIKGVSEVIQTV

SEQ ID NO: 31 - DNA, 147 bp, Artificial
  ORF16
atggaaatgaatttatttagccctaaatggtggattggttctattgtcacagcattcatgacaatgttctttatttacctaacgaaaaatattgcagcaaaagcaaacatcccgtttgtatcaaaagttactgaggaggcttacaag

SEQ ID NO: 32 - protein, 49 aa, Artificial
  Protein encoded by ORF16
MEMNLFSPKWWIGSIVTAFMTMFFIYLTKNIAAKANIPFVSKVTEEAYK

SEQ ID NO: 33 - DNA, 1062 bp, Artificial
  ORF17
atgggtcaacaacaacaattatctgcacaacaacgtgccgcatatttcggtacagcaacgagacagaactatcaaatgctaccagcacagcaagtcacacaagaaaatagcacggtagaatttaccctgccaaaagcacgtttattatcaaaaatttatttaaatgtagaagccgtagcgactctaaagagtaaagggacagccatccaaacgcacgacttctcaccatatactattttacgacgtgtatcactagacctcaacaacggattcagtcctttcattgtaagtggtcgagacttaatgcaatataacttgctgcgtttaaatccaaatgtattattcccagcttctacacctagaggtatgaactatatcgaaagtggggcatccgtggaaggtaaagatgcaaagattaaattcactgttgaattacctgtcacactaaaccaacgtgaccctgtaggacttgtgttattgcaaaatgctgaaacaagtgtaaccctaactgtcgatgttgcacagttagcaaatgcatatacattaaatgcttctaacacggatcaagttttatttaaatctatgaaagttgttccgatggtcgaaacgtttagcattccaccgattccagaagcattccctgacatttcaacactgaaactagtttctagtaaatcagatacatttgcgggtaatggacaaaacatcgtgaaattaaacacgggtacaatttaccgcaaaatgctgttgtactttgaagacaaggacgggaaaccgctagaagacacggatttccaagggaacattgagcttgtctttaaccaagccgatatcccttatagcatcaagcccgaaattctgtcacatatcaatcacagtcaattaggctatccattaccaaaaggattatatgctcttgacttcacgaatcaagggattccaaatttaggcggtagtcgtgactttattgattcagaacgtttaacagaattgtgggttcgcttctcaacattaaaagaaggaaaagtgacagtcgttagtgagaacttgtcacgcttacga

SEQ ID NO: 34 - protein, 354 aa, Artificial
  Protein encoded by ORF17
MGQQQQLSAQQRAAYFGTATRQNYQMLPAQQVTQENSTVEFTLPKARLLSKIYLNVEAVATLKSKGTAIQTHDFSPYTILRRVSLDLNNGFSPFIVSGRDLMQYNLLRLNPNVLFPASTPRGMNYIESGASVEGKDAKIKFTVELPVTLNQRDPVGLVLLQNAETSVTLTVDVAQLANAYTLNASNTDQVLFKSMKVVPMVETFSIPPIPEAFPDISTLKLVSSKSDTFAGNGQNIVKLNTGTIYRKMLLYFEDKDGKPLEDTDFQGNIELVFNQADIPYSIKPEILSHINHSQLGYPLPKGLYALDFTNQGIPNLGGSRDFIDSERLTELWVRFSTLKEGKVTVVSENLSRLR

SEQ ID NO: 35 - DNA, 222 bp, Artificial
  ORF18
atggctggagaaatgagtcattttatgaaagatgtatatccaaacatgggttttcaaaatacaacctatatatccattcccgaagcggaagaccaacaagcattagttgacgatcaaaagattgctgaagagtcggggaaaatggaaaacaaggcgggacataaaaatatcatgctggggattgtcttgatcttaattattatgttcgtactaggaaaggtg

SEQ ID NO: 36 - protein, 74 aa, Artificial
  Protein encoded by ORF18
MAGEMSHFMKDVYPNMGFQNTTYISIPEAEDQQALVDDQKIAEESGKMENKAGHKNIMLGIVLILIIMFVLGKV

SEQ ID NO: 37 - DNA, 169 bp, Artificial
  ORF19
atggaaatggaagtagcacaatttattagcaataacgggtttgcagcattcgtagccgtttttatgcttgtcaaaggttcaaaggacaaccaaaatatgacggctgccattaataaattagaaacagctattacattattaaatggaaagaaagtcgaggacgataaat

SEQ ID NO: 38 - protein, 56 aa, Artificial
  Protein encoded by ORF19
MEMEVAQFISNNGFAAFVAVFMLVKGSKDNQNMTAAINKLETAITLLNGKKVEDDK

SEQ ID NO: 39 - DNA, 471 bp, Artificial
  ORF20
atggatagagggttaacattcttcacattggcattgctgctgatatggctagtctttgacgacctttttgggaacaagaaatacttgtctaaattagcgggctctatgacaccgaatttatcccttcctgatcctgtacgggacatggtggataagaccgtggaagacacgaaagagaatgtgaagaaagatgttgctgacacgaaaaaggatacgaaagatgccattaacgatacaaagaaatcatgggatgactttgtgaatggtggctttgaaaaagaaatgaaaaaggatgtcaatgactttaaagattggatgaaagaccttcctaatcctgacaagatgaaaaagaaaaccaatgatgatttcaaagaaatatgggatgaaatcaataagtcattagaagacaccaaaaagtcagccgttgatatgtgggatgatgtcacatcatcggtgaaagggtggttcaaa

SEQ ID NO: 40 - protein, 157 aa, Artificial
  Protein encoded by ORF20
MDRGLTFFTLALLLIWLVFDDLFGNKKYLSKLAGSMTPNLSLPDPVRDMVDKTVEDTKENVKKDVADTKKDTKDAINDTKKSWDDFVNGGFEKEMKKDVNDFKDWMKDLPNPDKMKKKTNDDFKEIWDEINKSLEDTKKSAVDMWDDVTSSVKGWFK

SEQ ID NO: 41 - DNA, 174 bp, Artificial
  ORF21
atgaaagaatttacggagtcactcggatttattgtagcgtttatgaccatgacaattttcatgtctatgtttacgaatgaagccattacaaatggttttctcatgatcgtactcgcttctatgttggtactcaattccgagaagtttacaaaaatgttagatggggtgatgaaa

SEQ ID NO: 42 - protein, 58 aa, Artificial
  Protein encoded by ORF21
MKEFTESLGFIVAFMTMTIFMSMFTNEAITNGFLMIVLASMLVLNSEKFTKMLDGVMK

SEQ ID NO: 43 - DNA, 144 bp, Artificial
  ORF22
atgagtcgtgtactaggaattgtttcgggtattggtatgttaattgctgtttacctgtttttaaataatgcgaaacaaacaacgcaaatcattgacagcattgctgggaatggtgtgaagggtatcaaaacattacaaggtcga

SEQ ID NO: 44 - protein, 48 aa, Artificial
  Protein encoded by ORF22
MSRVLGIVSGIGMLIAVYLFLNNAKQTTQIIDSIAGNGVKGIKTLQGR

SEQ ID NO: 45 - DNA, 273 bp, Artificial
  ORF23
atgttagaaggcacgtatagaaatcgtgtgcataacggacaagccttgcaagcacttactaagcgggtagaaattcccgatgcccgtttaggtgtcggtgctgcggaacatttagaagcaatgggtatccgtgtgcaatatgaaaataaagtaccaaaactggtattaccttctgtacatcatttaccgttcgaacaagcacgacctactaaagtagaagccgatatgtttgtcacaaatgactttcaaattggtgacgccattatgggggtg

SEQ ID NO: 46 - protein, 91 aa, Artificial
  Protein encoded by ORF23
MLEGTYRNRVHNGQALQALTKRVEIPDARLGVGAAEHLEAMGIRVQYENKVPKLVLPSVHHLPFEQARPTKVEADMFVTNDFQIGDAIMGV

SEQ ID NO: 47 - DNA, 630 bp, Artificial
  ORF24
atggctgaaattgtaccagttggtgggggcgggcatggctcgtctccttctaaaaagaaaaataagaaaatgttatttcttgctggcggtgtaggtgttgttgttctacttgttttcttgcaacgttcgaaaggtggaagtagtagcgggaatgtagataccctgcaaaatacgattcctatttcggattcccaaaggctcgacaattttcaatctattgtgagcggggaaacatcggcacagatcaacggcatgatgaaagatgcacaggacggttggtcgggaatgttcaaggactttagcgaaaaaatgaccaatcaaatgaaagaaatggatgaacgcaacaaggaatattccaaacaacagcaagaatgggtgaaggattcctttttaaatattaaggactcgctaggtgtgggagcaattcgaaatgaagataacgcaatatttacggtgggtaaaggaacaacagggaaacagccgtataacgaacaattaaacgatttacgcaatgaccgccaaaagctgaacgaggaaatcaaacgtacacaatccgttattacctatcgtaaaaacaacggtctagacgtttccgctcaagtacagcactacaagaatttaggtgaattg

SEQ ID NO: 48 - protein, 210 aa, Artificial
  Protein encoded by ORF24
MAEIVPVGGGGHGSSPSKKKNKKMLFLAGGVGVVVLLVFLQRSKGGSSSGNVDTLQNTIPISDSQRLDNFQSIVSGETSAQINGMMKDAQDGWSGMFKDFSEKMTNQMKEMDERNKEYSKQQQEWVKDSFLNIKDSLGVGAIRNEDNAIFTVGKGTTGKQPYNEQLNDLRNDRQKLNEEIKRTQSVITYRKNNGLDVSAQVQHYKNLGEL

SEQ ID NO: 49 - DNA, 654 bp, Artificial
  ORF25
atggctgcggatattcgtccattcattgctgatgcacagcgaatccaaaaacaaacgggtatccccgcatctattatattaggtcaaatcatttttgaatcaagcgggaaaaaccccggtggtttatcaggacttgcctataacaacaagaaccttttcgggatcaaaggaaaaggaacggctgggacagcgaatatgtggtcaaaagaatatgatgcgggagggaatcgggtttctggtttccgctcgtataattcttatacagaatcactaaatgaccatgcaagattgctgcaaacagaccgctatgcaaagtatttaaaaaatgcgaaatcagtggatgactttgcaagagggattataaaaggtggttacgccacagatccaaattatgcaaaccaactattaggcattattaaatcaaacggacttactaaatacgatgatggaacatacacctttacgggtggtgatgtgtcgggcggttctgctggtggtggaggtggcggggtttccttctttgccccactatttaacgccattgtacgaggtctcttattctttgtatgtgtcgtggctgccttgctattattcgcaaaagcatttccaagcgtggaacaaactgtcaagtcaacagcgaagaaggtgaagtca

SEQ ID NO: 50 - protein, 218 aa, Artificial
  Protein encoded by ORF25
MAADIRPFIADAQRIQKQTGIPASIILGQIIFESSGKNPGGLSGLAYNNKNLFGIKGKGTAGTANMWSKEYDAGGNRVSGFRSYNSYTESLNDHARLLQTDRYAKYLKNAKSVDDFARGIIKGGYATDPNYANQLLGIIKSNGLTKYDDGTYTFTGGDVSGGSAGGGGGGVSFFAPLFNAIVRGLLFFVCVVAALLLFAKAFPSVEQTVKSTAKKVKS

SEQ ID NO: 51 - DNA, 525 bp, Artificial
  ORF26
atgagtggtactaatggtctcaaactaaacgataagctgcaagaagcctataacaaggcgattggtgcggggttacgttttacaagtggtttccgtcctggatcaaaaggaccgagcggaaaagccgatagccattcccaaggcatggcaatggactttgcagggaatcccgctcaaatggctgtctttgccgaatgggcgaaaaagtcgggtctcttttcagaagtgttatataaaacggctggacactacgatcatgtacatgtgggctggcaagaaaacaaacatcctgctgggaaagtatatgttggcgaccatacactcattgaccgtgtaggtggcgggactttaggtgacttgcaaacagtcggtgacacttctgcacctgctggtggcggagacaaggcgggtttcgtgtcttccctatttaacggcatatttcgagtattaatgattgtgatatgtctaatcggtggcgtgtacttctttatgaatgcattcccgcaaatgaaacaattaatcaaa

SEQ ID NO: 52 - protein, 175 aa, Artificial
  Protein encoded by ORF26
MSGTNGLKLNDKLQEAYNKAIGAGLRFTSGFRPGSKGPSGKADSHSQGMAMDFAGNPAQMAVFAEWAKKSGLFSEVLYKTAGHYDHVHVGWQENKHPAGKVYVGDHTLIDRVGGGTLGDLQTVGDTSAPAGGGDKAGFVSSLFNGIFRVLMIVICLIGGVYFFMNAFPQMKQLIK

SEQ ID NO: 53 - DNA, 900 bp, Artificial
  ORF27
ttggatagaaaaacaaatagtacgtggagggagcaaaccctcacattaccaccgaaaacggtgtatgacgttgtattccctgatacaaagccaaaccattaccatattaataatttatcacctgcaatgatttatttaggtgtatccattattgcatcaccacaatcctatgacattgccgtgacaggtaacggggacaacatacatgcacgtgacctcggtgcaactcgaatcactttatacaatgacagtcctgataaggctcgaattgttttaacatcgtttgaagataagtttaaccctgctgtgctttcaggtcgtggtagcgttacggtttcgggtggtggcggtggtggtgctggtggtgtcattactggtttcaacgcttcccttcctagcggtgataataatatcggtcgggtaaaaatttcggaaatgcctgcaattgattttgtactcggtacattacccgctggttcgaacaatataggtaaagtggaagttagcaaattaccaccacttgctagtgttggtgggaaaattggggatgtcggaatttcgggaggcgtgtcgattacctctatgccacctgtgcaagtaacaaatgatcctgtacgtagtacatgtcgtgcatggagtggtgcggttgatactacacatgtaacctttgatatggttgacggggatgtgttgaaattcagttatatatccaatgatggtgatacagacctattcattaattttgatgatgctaaagcaaaccccgataacttgaaaggtgcgggattatcaggaaagaatgcttctatatggttaaaacctggtgagatcattacagaattcactagaaagacaactaaggtgaacatgattaggaaatcaggtaacggtatcgttcga

SEQ ID NO: 54 - protein, 304 aa, Artificial
  Protein encoded by ORF27
LDRKTNSTWREQTLTLPPKTVYDVVFPDTKPNHYHINNLSPAMIYLGVSIIASPQSYDIAVTGNGDNIHARDLGATRITLYNDSPDKARIVLTSFEDKFNPAVLSGRGSVTVSGGGGGGAGGVITGFNASLPSGDNNIGRVKISEMPAIDFVLGTLPAGSNNIGKVEVSKLPPLASVGGKIGDVGISGGVSITSMPPVQVTNDPVRSTCRAWSGAVDTTHVTFDMVDGDVLKFSYISNDGDTDLFINFDDAKANPDNLKGAGLSGKNASIWLKPGEIITEFTRKTTKVNMIRKSGNGIVRILGV

SEQ ID NO: 55 - DNA, 399 bp, Artificial
  ORF28
atgggactgaaaaaacctagcggtgcaccctttatgtcagcgcagggggaatcgtttgtaacaatagaatcccaaaaatcagctacattagaaattaataatttatcattcacacctaatatgattgtgattagatgtgaaactcaatatggtgaggtctatcaaatgacctatcatccttttcaaatgatttacggtatcaatgtaacaagaggggtaaatgtagataaaaatcgccctacttatttatcaaatgattcttatgggttacttacaccaagtgcaaaaggatttaatgctaagttaactcctgacacggatatttttagaggtagcgcattatttaaatggaaagcttattatttcccttcagtaaaagaggaagcggaaaaaccattc

SEQ ID NO: 56 - protein, 133 aa, Artificial
  Protein encoded by ORF28
MGLKKPSGAPFMSAQGESFVTIESQKSATLEINNLSFTPNMIVIRCETQYGEVYQMTYHPFQMIYGINVTRGVNVDKNRPTYLSNDSYGLLTPSAKGFNAKLTPDTDIFRGSALFKWKAYYFPSVKEEAEKPF

SEQ ID NO: 57 - DNA, 354 bp, Artificial
  ORF29
atgtttgtaccattacggatttactacgataagaaaacagggttaatcattcaatacactggtaactttcaagataacaacatgattcaagcacctaccattgaagatgattttacgagttatcaatctttaaatgaaagagtgaaagagactgtaggtgttattgaactagaacaaaaccagtataaggaagaattatataaagcaacaaacgtgactgttgatgtaaagacaggtcaacttatgtttgattttacacctattgttaaaaaggaaatcgaagagaaaaagacactggaacaacgtatcacattagttgaaagtacaattaacgatattctattaggaggaatg

SEQ ID NO: 58 - protein, 118 aa, Artificial
  Protein encoded by ORF29
MFVPLRIYYDKKTGLIIQYTGNFQDNNMIQAPTIEDDFTSYQSLNERVKETVGVIELEQNQYKEELYKATNVTVDVKTGQLMFDFTPIVKKEIEEKKTLEQRITLVESTINDILLGGM

SEQ ID NO: 59 - DNA, 147 bp, Artificial
  ORF30
atgacagttttatctatgaaagtacgttacattttaaatcaatggttaatgggtggtttcaaagatagcgaccttccaacactggtagatcgtggacatattacagaagaacaacgcacttacttcttgtcattgaaggaggaaaaa

SEQ ID NO: 60 - protein, 49 aa, Artificial
  Protein encoded by ORF30
MTVLSMKVRYILNQWLMGGFKDSDLPTLVDRGHITEEQRTYFLSLKEEK

SEQ ID NO: 61 - DNA, 756 bp, Artificial
  ORF29
atgtatgatttatatacgtttggtgctggtcacaattttaaagtacccggggcaagtggaaacggatataaggaagaggtagagacaagacgggttgtaaaacggttgttagagatatgttaccagcatggtattaaagctgtagataccactgataatgatggtagaacacaacgtgagaatctaaacaacatcgttcgtaattgtaatagctaccctaaaaatggtcgtcttgatgtagctatccattttaaccaagcggaaagcgaaacaggtggtgttgaggtttggtattacgatcaagctggacttgctgcaaaggtaagtaaagatgtggctgctgcactcggtttacgtgaccgtggggctaaagaggggaaaggtcttgccgtactgaatggaacaaacgccccagctatattgatagagttgccgtttctatcacatgtagggaatatgcaagcgtatgaatccaatttcgagcctatgtgtagagccattgtacaagccgtgacggggcaacaggttgcggtgaatgggatacgtcctgttagcaagaatgttattcagacgggggcattctcaccgtatgaagcgactgatgcaatgacggcattaacgtcactaaaaatgacaggggtgttctatttacaagcaaatgggttaccatatattgtcacagacccgacatcggatgcacagttagaagctgcaaaagaatatttcaaaagaaaagattggtggtttgatgttaag

SEQ ID NO: 62 - protein, 252 aa, Artificial
  Protein encoded by ORF31
MYDLYTFGAGHNFKVPGASGNGYKEEVETRRVVKRLLEICYQHGIKAVDTTDNDGRTQRENLNNIVRNCNSYPKNGRLDVAIHFNQAESETGGVEVWYYDQAGLAAKVSKDVAAALGLRDRGAKEGKGLAVLNGTNAPAILIELPFLSHVGNMQAYESNFEPMCRAIVQAVTGQQVAVNGIRPVSKNVIQTGAFSPYEATDAMTALTSLKMTGVFYLQANGLPYIVTDPTSDAQLEAAKEYFKRKDWWFDVK

SEQ ID NO: 63 - DNA, 14398 bp, Artificial
  AP50 Genome
catggtgttcagcccatctaataaattaatagggtagaagtattgacatacatataaatttatgaaacacgatattaatttccaattgacaattttccaatttaccaacttaccaattttagaaaatttcctattggaaagggtggaaaagtgtgacattattttttactagtgatataatagtgacagaacaaattcgaacatcctttatgtcatcagactaagtgttcggttttctgtaacataactgtaacattccaattattgcacatctacccaatacatgctaagataggtcttgtaaggacaacacaggacaatgaagaacactaagtgatagggtgtattcattaaaggatgtggggagatgaagtagaatgctattctacactgtaaaggaatttgcggaaatggctaagatttcagagaagactgtgactaggtacatcaagacgggtgatttggaagcagtcaagtttggcggacaatggcgtatcactgaaacagcggtacaaagctatattaaaaataattcaaatatcggaggacgagaaaatgactaataaaaatgaacaattaaataatggtgctgtaacatcttatgtggatactgacatgcttgctaatggggaacaaccaatggtaattactcctgaagtaaatacaaatgacatcgtgacagtggacaaggtgtcaccatctattttcgaagctcaaggcggtaaggatgtattcttttcttctattcaaacgaaagaccgtaaatcagctatcaaagtctacaacgctatcaattctagcgaaaatcctttagcagatcatcgtggtgaagtgttacatatcactgacatggttgcacatgcaattacattagaagatgatgttacaaaagaagatgtggacgcattacgtgtcgtattagtggacaaggacggtaaagcataccatgcaatttcacaaggtgttgtgtcatctattcaaaagattattagcatcgttggaccagcaccatggacagacgagccacttgaaatcgtacctgtagaagtgaagacacgtaaaggattcaaaacattaactctacaattacaaggttaatttcgataacaagtgaatgagggtacacattagtgtatcctcttttttctaaaagaaagggtgcttgctagtggatggtataaaagaagtggtacaagtttctattaaaaccaatgagcgtgacatcgaattcacatcatttgcaggattgaatcaaattaaacaatcgcttgatggtgatgtgtcatcggtcgtattaacggatgaagaactagaaatgatgttatgtgtgaagcagcgtgataagttagcatcattcttccaatcggtgctagaacgtaagaatgcggtataaggctttaaaacctgtgacatacgtgtcgcaggtttaaacagctttataaggtaggtgagaacatggctaataaacgtcaacgtaaaaagatagtgaaaaagaaacaagaatcctttttatcatcggtgggttattcgaagaaacaaatgaaaacgattagcacaactgatagagcaaaggtagtaaagaaagaaacatataaaaagaaaaagcgtgacaagtatcaccaagcaagatcaatggggtttggttctaaagaagcaaacaaaatgagtagttggtctgactcacgctttataaagtatatagaagaatttaattcttactatatgattgtcatgtataaagatgtaacagaagagacggacagcgaagcattacatatgattaagaaccacacgaaaagacgtagcacatccaacttacttagaagtattaaaggatggttgtctgtagataaaaatcaaggttatataggtggatatgaaattcaagtaggtaagaaagacgaaatagatttccatttatatgcttataaacagcgaaagtatttacaagcatatagaggacaagggttacaattaaaacctctattaaacttattagaaaatatgatggtattattatacatggtagaagctaaagaccaatttgttgaggacttatgtacaaacttgagaaagctaccatatgaacaggcacatataaacgctaattatatagaagaagaatttataacagatagaagtgacttgcatttttaggaggtgcgatatagtgagtaataaacaaaaaaaagagcgtcaaaagcctgcgaagcttttaacgctggacacggaaacacgaggtttgacgggcaacgtgtttcgtgtcggattgtttgacggtacaaattactataaatcaaatacctttgacgagattcttgatttatttgaacagtataaagattatgagtgtcacgtatacgtccataatttagatttcgatttagctaaaattgcaactactctatttaaacgtgatagggtgcggttcgctaaatccatctttattaatggtaacgttgtgacattacattctgactctatgatactacatgatagccttagattgctacctggaagtttagaaaaattatgtaaggatttcggattaaccgacaatgcaaagaaagatttgtcggaagttatcaaagaacagggatatgcggtatataaaaaagatggtgtgacgtttgataaaaagaaatcgttaggtaactattttgaaaacgtaccagctgacgatccaacgctaaatgagtatttagaatttgactgtcgctcactgtatgaaatattaacaattgttatggacatagctaatataggtcttgaaacacttgtgatgtgtccaacaacagcttctcttgctatgagagtgtataaggaacaatatcgtgaacagtatgataaagtagcaacacatttttatatgggtgaatggggacaatttttagaagagcatgtacgacaatcctattatggaggtcgtacagaagtatttacaccacacttaccacatggttatcactatgacgtaaacagtttatatccgtatgtcatgaaaattgcaaagtttcctgtaggctatccaaaccttttaaaagatggacaagccgcgacgaaatggaaacactggaaacgtagagcaataggtgggggtgttatgtggtgtcgtgtagatgtacccgaggatatgtatatacctgtattacctaaacgtgacccgagcgggaaactattattccctgtaggtaagctagaaggtgtatggacattaccagagttattagaagctgaaaagaatggttgcacgatagaagcaatctatcaaatggtatattgggaacacatggaaccgatattcaaagagtttgttgagcattttgaggacttgaaaaagaactctaaaggagcaaaacgaacatttgcaaaacttattcagaatagcttgtatgggaagtttggtatgaatagggtgcgtgtcagtttgggggacatggaagaccgctatgatctacacgaaaagcaaataccatataaagaatttaaacatgattgtaatgggttaacactagaatttattcaatatataagcgaaagtaaggctagttacatacaacctcatattgcaacctatgtgacagcctatgcacgtatccttttatttagagggttaaaggaacaagctagtaaaggtgtgctaggatactgtgacacggattctattgcaggtacggcaaaaatgcctgatgaaatgattcatgatgaagattatggtaagtgggcgttagaaggtgaactagaagaaggtatattcttgcaacccaaattctatgcggaacgctatacaaacggaaaagaagtcattaaggcaaaaggaatacctcgtgagaaaatggaagagttgtcttttgagaattacaaggaatggcttgaaatcatgaaagaagggcaacaggaacgtatcgacattttcgaaggatatgagtcacgcaagaaattttcaacaacattaaaagcatctgaggattttgacacattacgtgaaatgaagaaatctattaatttattattagaacaaaagcgtgacattgattataaagggaatgtaacaagacctcataaacgttacgattacggggataagaaggacaagattgattatgaagattataagtctagagaagataaattaaacaatatgtatgatgatgtagacgatctaaaagagcaagtggacgaaataggttacatcaaatgtatgaaacaaggtgacatgtattttgaggaatacaaacatttaacaaagtcagttaaaagtaaatattttcgtagaacggggacacctatagacgtatgggcgaatgagtcgggatgggatgttaacgaattactagaagaattacgattgatgggggtatgttaaaatgttaaccgatagagaacaagaagcgttagcgtgtataagcgggtatatgagacaaaacgggtttgcaccatccgtgcgagaaatggctggtttattatttgtcagtcacaagaccgcacatcgttatatgattcaattagaaaccaaaggacacattaagagaatacatcatcgttcacgtgctatccaactatgtgtataaaaggaggaatagagcatgcgtgataaagttttagacttaatcattgaactatccaaatcaacaaaacaggtcgtagcaaaagatttcattattaatgaattatataaaatagcaaaagaagatgaatcgaaagagaaggagactagcaaataaatgctagtctttttatttgtccaaaatgacacatttgtgacgttacaagcgaacatatgtttgggttatacttgtcacatcaagcaataaggagtgacaaaaatggaaggtattgatttaagtcatgtgcaatgttcattgccacctattccaaacccgttaacgtttgaagatttaacagaagaacaatttaaagcgttattaaatgttattcatgattttaaattcgcttgtagagaaagtaatttaccacttgctttctattatgtattagaaaaatgtactgaccctgtagtgaaacaagaattaatagatgctcatagatatgggtgttaaggagtgtacaagatgttcaaaacattatcaaaactgtaccgtgacttattacatcaaaacatagatttgcacaatgaaaatacaaaactacgattacaaaatgcacggctgcaaagtaagttagcaaccgctgaaatagatttataccattttaaaaattcaatagaaaggatgattaacaaatgacagactcattacaagtagtggaagaaaaaacgaaatttagattaggagattttcaattctttgcaaagaagaaagaagaggacgatcaagaggaagaagaggaacttgaggaagaggaagaagaggaagaagaaaagccgaaaccaaaacgtaaatcaaaaagcggggaggatgcaccagcatgggcacaagaaataatcggactgttgaaaccgaaagcggaggaacagcaaacaaaacagaaagtaccagtacccgaagcaccaatagtggaggacgaggaagaggaagaaccgccgaaagccagtccactgaaaagcttcctaagtcggttgtggtagaagtcccgaacggtgagaagtcacccgaggacaccaaaaaggaagaacaacgaaaggcagcagccgcacgaaaacgtaaatcacgtgcagcagccgcaacgaaaaagaagtcgtcaccatctattggtgatgcaacgcaattaaaagtattgcttcttactacgtcacagatcatagcagcaagagaaggtatgagcgtatgggcgatgacggaacaagaggttgaccaaattgttacaccgctttatagcatcctatctaaaaatgatggggtggggcaagtcatgggtgaatatgccgaccacattgctttaatcgtggcagcatttactatatttgtaccaaaatttatgatgtggaaagcatcaagacctaagaaggagggaacgcactatgctagaccaaatccaaattccaaacgagaacaaggaaagcaaacaggagaggttgcaactagtagtagaccaagtggtggacagcctaccaacaacggtacgacttttggcgggcagctatctgaactcgttccgccaagtgctggaatctgaacagcatgacattgacggaaacattgatttagccttgtcacgtttacgtgagtacatcgactatatccaatatggtcacgatcaagaaaatgagtaggtgaagacatggaacgtatcccaaaagaccaacacgtatttatcacgggacaaacgggtacggggaaatcttttcttgctgaaacgtatttagcaggttacgaacatgtaattaagttagatacaaaaggtgaggtgtttgaaagacgaaaaaagaaacagcctgtatggcgtgggttacgtgaaggaaaggactttacagtcatagagcatttagacgaaatcgacagcgtggaaacaaagaaaatcatttatgcacctgtctttcaagagcaagaaatggaatactatgatgctctcatgcaatgggtgtacaggagagaaaatacacaattatggattgatgaactcatggaagtatgcccgagtcctttcaaataccctccctacctcaaaggtcttatgactagaggacgttcaaaagaagctactgtgtgggcgtgtacgcaacgcccgagtgacattccttctattgtaatggggaatagtgaccactttttcgtctttgaccaaaacttgccacaagatcgtaagaagttatgtgagacaacgggtagttataagtttatggaattaccgggctatcgtaacttttggtattttaagcgtggcatgacagatcccgtactcgccacattgaaattatgatccttgaaagggggtgtttaggtggaggggaaatttgcagggattggactgaaaaatattttagccatcttctttttattcattgtatttatagttgttgctaaagtaattttcacgaagtatcccataaaaggcgttagtgaagtaatacaaactgtataggaggaatggaaatgaatttatttagccctaaatggtggattggttctattgtcacagcattcatgacaatgttctttatttacctaacgaaaaatattgcagcaaaagcaaacatcccgtttgtatcaaaagttactgaggaggcttacaagtaatgggtcaacaacaacaattatctgcacaacaacgtgccgcatatttcggtacagcaacgagacagaactatcaaatgctaccagcacagcaagtcacacaagaaaatagcacggtagaatttaccctgccaaaagcacgtttattatcaaaaatttatttaaatgtagaagccgtagcgactctaaagagtaaagggacagccatccaaacgcacgacttctcaccatatactattttacgacgtgtatcactagacctcaacaacggattcagtcctttcattgtaagtggtcgagacttaatgcaatataacttgctgcgtttaaatccaaatgtattattcccagcttctacacctagaggtatgaactatatcgaaagtggggcatccgtggaaggtaaagatgcaaagattaaattcactgttgaattacctgtcacactaaaccaacgtgaccctgtaggacttgtgttattgcaaaatgctgaaacaagtgtaaccctaactgtcgatgttgcacagttagcaaatgcatatacattaaatgcttctaacacggatcaagttttatttaaatctatgaaagttgttccgatggtcgaaacgtttagcattccaccgattccagaagcattccctgacatttcaacactgaaactagtttctagtaaatcagatacatttgcgggtaatggacaaaacatcgtgaaattaaacacgggtacaatttaccgcaaaatgctgttgtactttgaagacaaggacgggaaaccgctagaagacacggatttccaagggaacattgagcttgtctttaaccaagccgatatcccttatagcatcaagcccgaaattctgtcacatatcaatcacagtcaattaggctatccattaccaaaaggattatatgctcttgacttcacgaatcaagggattccaaatttaggcggtagtcgtgactttattgattcagaacgtttaacagaattgtgggttcgcttctcaacattaaaagaaggaaaagtgacagtcgttagtgagaacttgtcacgcttacgataagaagaggggatttccccttttcttctatataaggagggacattgcatggctggagaaatgagtcattttatgaaagatgtatatccaaacatgggttttcaaaatacaacctatatatccattcccgaagcggaagaccaacaagcattagttgacgatcaaaagattgctgaagagtcggggaaaatggaaaacaaggcgggacataaaaatatcatgctggggattgtcttgatcttaattattatgttcgtactaggaaaggtgtgagtctaatggaaatggaagtagcacaatttattagcaataacgggtttgcagcattcgtagccgtttttatgcttgtcaaaggttcaaaggacaaccaaaatatgacggctgccattaataaattagaaacagctattacattattaaatggaaagaaagtcgaggacgataaatgagcaaaacattaattctcgtggttgcgattttctgtttatggtttttcgtaatcaagaaaaagaaagcgtgatgtaaatggatagagggttaacattcttcacattggcattgctgctgatatggctagtctttgacgacctttttgggaacaagaaatacttgtctaaattagcgggctctatgacaccgaatttatcccttcctgatcctgtacgggacatggtggataagaccgtggaagacacgaaagagaatgtgaagaaagatgttgctgacacgaaaaaggatacgaaagatgccattaacgatacaaagaaatcatgggatgactttgtgaatggtggctttgaaaaagaaatgaaaaaggatgtcaatgactttaaagattggatgaaagaccttcctaatcctgacaagatgaaaaagaaaaccaatgatgatttcaaagaaatatgggatgaaatcaataagtcattagaagacaccaaaaagtcagccgttgatatgtgggatgatgtcacatcatcggtgaaagggtggttcaaataatgaaagaatttacggagtcactcggatttattgtagcgtttatgaccatgacaattttcatgtctatgtttacgaatgaagccattacaaatggttttctcatgatcgtactcgcttctatgttggtactcaattccgagaagtttacaaaaatgttagatggggtgatgaaataatgagtcgtgtactaggaattgtttcgggtattggtatgttaattgctgtttacctgtttttaaataatgcgaaacaaacaacgcaaatcattgacagcattgctgggaatggtgtgaagggtatcaaaacattacaaggtcgataaggagggtgtgacatgttagaaggcacgtatagaaatcgtgtgcataacggacaagccttgcaagcacttactaagcgggtagaaattcccgatgcccgtttaggtgtcggtgctgcggaacatttagaagcaatgggtatccgtgtgcaatatgaaaataaagtaccaaaactggtattaccttctgtacatcatttaccgttcgaacaagcacgacctactaaagtagaagccgatatgtttgtcacaaatgactttcaaattggtgacgccattatgggggtgtaaagaatggctgaaattgtaccagttggtgggggcgggcatggctcgtctccttctaaaaagaaaaataagaaaatgttatttcttgctggcggtgtaggtgttgttgttctacttgttttcttgcaacgttcgaaaggtggaagtagtagcgggaatgtagataccctgcaaaatacgattcctatttcggattcccaaaggctcgacaattttcaatctattgtgagcggggaaacatcggcacagatcaacggcatgatgaaagatgcacaggacggttggtcgggaatgttcaaggactttagcgaaaaaatgaccaatcaaatgaaagaaatggatgaacgcaacaaggaatattccaaacaacagcaagaatgggtgaaggattcctttttaaatattaaggactcgctaggtgtgggagcaattcgaaatgaagataacgcaatatttacggtgggtaaaggaacaacagggaaacagccgtataacgaacaattaaacgatttacgcaatgaccgccaaaagctgaacgaggaaatcaaacgtacacaatccgttattacctatcgtaaaaacaacggtctagacgtttccgctcaagtacagcactacaagaatttaggtgaattgtaatggctgcggatattcgtccattcattgctgatgcacagcgaatccaaaaacaaacgggtatccccgcatctattatattaggtcaaatcatttttgaatcaagcgggaaaaaccccggtggtttatcaggacttgcctataacaacaagaaccttttcgggatcaaaggaaaaggaacggctgggacagcgaatatgtggtcaaaagaatatgatgcgggagggaatcgggtttctggtttccgctcgtataattcttatacagaatcactaaatgaccatgcaagattgctgcaaacagaccgctatgcaaagtatttaaaaaatgcgaaatcagtggatgactttgcaagagggattataaaaggtggttacgccacagatccaaattatgcaaaccaactattaggcattattaaatcaaacggacttactaaatacgatgatggaacatacacctttacgggtggtgatgtgtcgggcggttctgctggtggtggaggtggcggggtttccttctttgccccactatttaacgccattgtacgaggtctcttattctttgtatgtgtcgtggctgccttgctattattcgcaaaagcatttccaagcgtggaacaaactgtcaagtcaacagcgaagaaggtgaagtcatgagtggtactaatggtctcaaactaaacgataagctgcaagaagcctataacaaggcgattggtgcggggttacgttttacaagtggtttccgtcctggatcaaaaggaccgagcggaaaagccgatagccattcccaaggcatggcaatggactttgcagggaatcccgctcaaatggctgtctttgccgaatgggcgaaaaagtcgggtctcttttcagaagtgttatataaaacggctggacactacgatcatgtacatgtgggctggcaagaaaacaaacatcctgctgggaaagtatatgttggcgaccatacactcattgaccgtgtaggtggcgggactttaggtgacttgcaaacagtcggtgacacttctgcacctgctggtggcggagacaaggcgggtttcgtgtcttccctatttaacggcatatttcgagtattaatgattgtgatatgtctaatcggtggcgtgtacttctttatgaatgcattcccgcaaatgaaacaattaatcaaatgaggtgaaaacattggatagaaaaacaaatagtacgtggagggagcaaaccctcacattaccaccgaaaacggtgtatgacgttgtattccctgatacaaagccaaaccattaccatattaataatttatcacctgcaatgatttatttaggtgtatccattattgcatcaccacaatcctatgacattgccgtgacaggtaacggggacaacatacatgcacgtgacctcggtgcaactcgaatcactttatacaatgacagtcctgataaggctcgaattgttttaacatcgtttgaagataagtttaaccctgctgtgctttcaggtcgtggtagcgttacggtttcgggtggtggcggtggtggtgctggtggtgtcattactggtttcaacgcttcccttcctagcggtgataataatatcggtcgggtaaaaatttcggaaatgcctgcaattgattttgtactcggtacattacccgctggttcgaacaatataggtaaagtggaagttagcaaattaccaccacttgctagtgttggtgggaaaattggggatgtcggaatttcgggaggcgtgtcgattacctctatgccacctgtgcaagtaacaaatgatcctgtacgtagtacatgtcgtgcatggagtggtgcggttgatactacacatgtaacctttgatatggttgacggggatgtgttgaaattcagttatatatccaatgatggtgatacagacctattcattaattttgatgatgctaaagcaaaccccgataacttgaaaggtgcgggattatcaggaaagaatgcttctatatggttaaaacctggtgagatcattacagaattcactagaaagacaactaaggtgaacatgattaggaaatcaggtaacggtatcgttcgaatactgggggtgtaatagatgggactgaaaaaacctagcggtgcaccctttatgtcagcgcagggggaatcgtttgtaacaatagaatcccaaaaatcagctacattagaaattaataatttatcattcacacctaatatgattgtgattagatgtgaaactcaatatggtgaggtctatcaaatgacctatcatccttttcaaatgatttacggtatcaatgtaacaagaggggtaaatgtagataaaaatcgccctacttatttatcaaatgattcttatgggttacttacaccaagtgcaaaaggatttaatgctaagttaactcctgacacggatatttttagaggtagcgcattatttaaatggaaagcttattatttcccttcagtaaaagaggaagcggaaaaaccattctaaaaggagggaactatatgtttgtaccattacggatttactacgataagaaaacagggttaatcattcaatacactggtaactttcaagataacaacatgattcaagcacctaccattgaagatgattttacgagttatcaatctttaaatgaaagagtgaaagagactgtaggtgttattgaactagaacaaaaccagtataaggaagaattatataaagcaacaaacgtgactgttgatgtaaagacaggtcaacttatgtttgattttacacctattgttaaaaaggaaatcgaagagaaaaagacactggaacaacgtatcacattagttgaaagtacaattaacgatattctattaggaggaatgtaaaatgacagttttatctatgaaagtacgttacattttaaatcaatggttaatgggtggtttcaaagatagcgaccttccaacactggtagatcgtggacatattacagaagaacaacgcacttacttcttgtcattgaaggaggaaaaatagtatgtatgatttatatacgtttggtgctggtcacaattttaaagtacccggggcaagtggaaacggatataaggaagaggtagagacaagacgggttgtaaaacggttgttagagatatgttaccagcatggtattaaagctgtagataccactgataatgatggtagaacacaacgtgagaatctaaacaacatcgttcgtaattgtaatagctaccctaaaaatggtcgtcttgatgtagctatccattttaaccaagcggaaagcgaaacaggtggtgttgaggtttggtattacgatcaagctggacttgctgcaaaggtaagtaaagatgtggctgctgcactcggtttacgtgaccgtggggctaaagaggggaaaggtcttgccgtactgaatggaacaaacgccccagctatattgatagagttgccgtttctatcacatgtagggaatatgcaagcgtatgaatccaatttcgagcctatgtgtagagccattgtacaagccgtgacggggcaacaggttgcggtgaatgggatacgtcctgttagcaagaatgttattcagacgggggcattctcaccgtatgaagcgactgatgcaatgacggcattaacgtcactaaaaatgacaggggtgttctatttacaagcaaatgggttaccatatattgtcacagacccgacatcggatgcacagttagaagctgcaaaagaatatttcaaaagaaaagattggtggtttgatgttaagtagacctagcgaaaagctaggtttttattttggaatttaatatatcgtaaatcatagatgtttattattgtcaatagggaatttagtcagatgggctgaacaccatg

SEQ ID NO: 64 - DNA, 24 bp, Artificial
  Synthetic Primer 606 AP50-left end off
atgtcacacttttccaccctttcc

SEQ ID NO: 65 - DNA, 23 bp, Artificial
  Synthetic 607 AP50-right end off
ccatatattgtcacagacccgac

SEQ ID NO: 66 - DNA, 24 bp, Artificial
  Synthetic 608 AP50-gap1 for
aaagacgtagcacatccaacttac

SEQ ID NO: 67 - DNA, 24 bp, Artificial
  Synthetic 609 AP50-gap1 rev
tctcaagtttgtacataagtcctc

SEQ ID NO: 68 - DNA, 24 bp, Artificial
  Synthetic 610 AP50-gap2 for
ggaaatcaggtaacggtatcgttc

SEQ ID NO: 69 - DNA, 24 bp, Artificial
  Synthetic 611 AP50-gap2 rev
acccctcttgttacattgataccg

SEQ ID NO: 70 - DNA, 32 bp, Artificial
  Synthetic 661 AP50_1_2000_FOR
catggtgttcagcccatctaataaattaatag

SEQ ID NO: 71 - DNA, 32 bp, Artificial
  662 AP50_1_2000_REV
gtttaatagaggttttaattgtaacccttgtc

SEQ ID NO: 72 - DNA, 32 bp, Artificial
  Synthetic 663 AP50_2000_4000_FOR
acagcgaaagtatttacaagcatatagaggac

SEQ ID NO: 73 - DNA, 32 bp, Artificial
  Synthetic 664 AP50_2000_4000_REV
atttcacgtaatgtgtcaaaatcctcagatgc

SEQ ID NO: 74 - DNA, 32 bp, Artificial
  665 AP50_4000_6000_FOR
ctgaggattttgacacattacgtgaaatgaag

SEQ ID NO: 75 - DNA, 32 bp, Artificial
  Synthetic 666 AP50_4000_6000_REV
ggtctagcatagtgcgttccctccttcttagg

SEQ ID NO: 76 - DNA, 32 bp, Artificial
  Synthetic 667 AP50_6000_8000_FOR
gaacgcactatgctagaccaaatccaaattcc

SEQ ID NO: 77 - DNA, 31 bp, Artificial
  Synthetic 668 AP50_6000_8000_REV
ggaaatccgtgtcttctagcggtttcccgtc

SEQ ID NO: 78 - DNA, 32 bp, Artificial
  Synthetic 669 AP50_8000_10000_FOR
acacggatttccaagggaacattgagcttgtc

SEQ ID NO: 79 - DNA, 32 bp, Artificial
  Synthetic 670 AP50_8000_10000_REV
tgcaagaaaacaagtagaacaacaacacctac

SEQ ID NO: 80 - DNA, 32 bp, Artificial
  Synthetic 671 AP50_10000_12000_FOR
gttctacttgttttcttgcaacgttcgaaagg

SEQ ID NO: 81 - DNA, 32 bp, Artificial
  Synthetic 672 AP50_10000_12000_REV
tatcttcaaacgatgttaaaacaattcgagcc

SEQ ID NO: 82 - DNA, 32 bp, Artificial
  Synthetic 673 AP50_12000_14000_FOR
atcgtttgaagataagtttaaccctgctgtgc

SEQ ID NO: 83 - DNA, 32 bp, Artificial
  Synthetic 674 AP50_12000_14000_REV
tgtgatagaaacggcaactctatcaatatagc

SEQ ID NO: 84 - DNA, 32 bp, Artificial
  Synthetic 675 AP50_14000_14500_FOR
gttgccgtttctatcacatgtagggaatatgc

SEQ ID NO: 85 - DNA, 31 bp, Artificial
  Synthetic 676 AP50_14000_14500_REV
catggtgttcagcccatctgactaaattccc

SEQ ID NO: 86 - DNA, 32 bp, Artificial
  Synthetic 731 AP50_651_682_FOR
tcctgaagtaaatacaaatgacatcgtgacag

SEQ ID NO: 87 - DNA, 32 bp, Artificial
  Synthetic 732 AP50_1350_1381_FOR
catcattcttccaatcggtgctagaacgtaag

SEQ ID NO: 88 - DNA, 32 bp, Artificial
  Synthetic 733 AP50_1349_1318_REV
ctaacttatcacgctgcttcacacataacatc

SEQ ID NO: 89 - DNA, 32 bp, Artificial
  Synthetic 734 AP50_650_619_REV
gtaattaccattggttgttccccattagcaag

SEQ ID NO: 90 - DNA, 32 bp, Artificial
  Synthetic 735 AP50_2576_2607_FOR
gtcggaagttatcaaagaacagggatatgcgg

SEQ ID NO: 91 - DNA, 32 bp, Artificial
  Synthetic 736 AP50_2607_2576_REV
ccgcatatccctgttctttgataacttccgac

SEQ ID NO: 92 - DNA, 31 bp, Artificial
  Synthetic 737 AP50_3258_3288_FOR
gaagcaatctatcaaatggtatattgggaac

SEQ ID NO: 93 - DNA, 31 bp, Artificial
  Synthetic 738 AP50_3288_3258_REV
gttcccaatataccatttgatagattgcttc

SEQ ID NO: 94 - DNA, 32 bp, Artificial
  Synthetic 739 AP50_4646_4677_FOR
cattgaactatccaaatcaacaaaacaggtcg

SEQ ID NO: 95 - DNA, 32 bp, Artificial
  Synthetic 740 AP50_4677_4646_REV
cgacctgttttgttgatttggatagttcaatg

SEQ ID NO: 96 - DNA, 33 bp, Artificial
  Synthetic 741 AP50_5355_5387_FOR
aagaggacgatcaagaggaagaagaggaacttg

SEQ ID NO: 97 - DNA, 33 bp, Artificial
  Synthetic 742 AP50_5387_5355_REV
caagttcctcttcttcctcttgatcgtcctctt

SEQ ID NO: 98 - DNA, 30 bp, Artificial
  Synthetic 743 AP50_6687_6716_FOR
agaagctactgtgtgggcgtgtacgcaacg

SEQ ID NO: 99 - DNA, 29 bp, Artificial
  Synthetic 744 AP50_6716_6687_RE
cgttgcgtacacgcccacacagtagcttc

SEQ ID NO: 100 - DNA, 31 bp, Artificial
  Synthetic 745 AP50_7338_7368_FOR
gcacggtagaatttaccctgccaaaagcacg

SEQ ID NO: 101 - DNA, 31 bp, Artificial
  Synthetic 746 AP50_7368_7338_REV
cgtgcttttggcagggtaaattctaccgtgc

SEQ ID NO: 102 - DNA, 34 bp, Artificial
  Synthetic 747 AP50_8713_8746_FOR
ggaaagaaagtcgaggacgataaatgagcaaaac

SEQ ID NO: 103 - DNA, 34 bp, Artificial
  Synthetic 748 AP50_8746_8713_REV
gttttgctcatttatcgtcctcgactttctttcc

SEQ ID NO: 104 - DNA, 32 bp, Artificial
  Synthetic 749 AP50_9362_9393_FOR
cgaatgaagccattacaaatggttttctcatg

SEQ ID NO: 105 - DNA, 32 bp, Artificial
  Synthetic 750 AP50_9393_9362_REV
catgagaaaaccatttgtaatggcttcattcg

SEQ ID NO: 106 - DNA, 32 bp, Artificial
  Synthetic 751 AP50_10693_10724_FOR
cgggatcaaaggaaaaggaacggctgggacag

SEQ ID NO: 107 - DNA, 32 bp, Artificial
  Synthetic 752 AP50_10724_10693_REV
ctgtcccagccgttccttttcctttgatcccg

SEQ ID NO: 108 - DNA, 32 bp, Artificial
  Synthetic 753 AP50_11356_11387_FOR
cccgctcaaatggctgtctttgccgaatgggc

SEQ ID NO: 109 - DNA, 32 bp, Artificial
  Synthetic 754 AP50_11387_11356_REV
gcccattcggcaaagacagccatttgagcggg

SEQ ID NO: 110 - DNA, 32 bp, Artificial
  Synthetic 755 AP50_12679_12710_FOR
gtcagcgcagggggaatcgtttgtaacaatag

SEQ ID NO: 111 - DNA, 32 bp, Artificial
  Synthetic 756 AP50_12710_12679_REV
ctattgttacaaacgattccccctgcgctgac

SEQ ID NO: 112 - DNA, 32 bp, Artificial
  Synthetic 757 AP50_13339_13370_FOR
gaagagaaaaagacactggaacaacgtatcac

SEQ ID NO: 113 - DNA, 32 bp, Artificial
  Synthetic 758 AP50_13370_13339_REV
gtgatacgttgttccagtgtctttttctcttc

SEQ ID NO: 114 - DNA, 24 bp, Artificial
  Synthetic 857 AP50-2235-2212
gtttccgtgtccagcgttaaaagc

SEQ ID NO: 115 - DNA, 26 bp, Artificial
  Synthetic 858 AP50-3764-3789
cccaaattctatgcggaacgctatac

SEQ ID NO: 116 - DNA, 26 bp, Artificial
  Synthetic 859 AP50-4183-4157
tcgtccacttgctcttttagatcgtc

SEQ ID NO: 117 - DNA, 23 bp, Artificial
  Synthetic 860 AP50-5771-5793
gagaaggtatgagcgtatgggcg

SEQ ID NO: 118 - DNA, 23 bp, Artificial
  Synthetic 861 AP50-6215-6193
cgtgaccatattggatatagtcg

SEQ ID NO: 119 - DNA, 27 bp, Artificial
  Synthetic 862 AP50-7776-7803
ctatgaaagttgttccgatggtcgaaa

SEQ ID NO: 120 - DNA, 27 bp, Artificial
  Synthetic 863 AP50-8228-8202
ctgtcacttttccttcttttaatgttg

SEQ ID NO: 121 - DNA, 26 bp, Artificial
  Synthetic 864 AP50-9771-9796
catttaccgttcgaacaagcacgacc

SEQ ID NO: 122 - DNA, 26 bp, Artificial
  Synthetic 865 AP50-10226-10201
ggaatattccttgttgcgttcatcca

SEQ ID NO: 123 - DNA, 25 bp, Artificial
  Synthetic 866 AP50-11771-11795
cctgatacaaagccaaaccattacc

SEQ ID NO: 124 - DNA, 26 bp, Artificial
  Synthetic 867 AP50-12229-12204
cccaccaacactagcaagtggtggta

SEQ ID NO: 125 - DNA, 22 bp, Artificial
  Synthetic 868 AP50_13761-13782
ggtcgtcttgatgtagctatcc

SEQ ID NO: 126 - DNA, 24 bp, Artificial
  Synthetic 869 AP50-14203-14180
ggtaacccatttgcttgtaaatag

SEQ ID NO: 127 - DNA, 23 bp, Artificial
  Synthetic 795 AP50_1229_FOR
ttcacatcatttgcaggattgaa

SEQ ID NO: 128 - DNA, 21 bp, Artificial
  Synthetic 796 AP50_1378_REV
acgttctagcaccgattggaa

SEQ ID NO: 129 - DNA, 24 bp, Artificial
  Synthetic 797 AP50_5749_FOR
acgtcacagatcatagcagcaaga

SEQ ID NO: 130 - DNA, 21 bp, Artificial
  Synthetic 798 AP50_5898_REV
agcaatgtggtcggcatattc

SEQ ID NO: 131 - DNA, 21 bp, Artificial
  Synthetic 799 AP50_7278_FOR
ccagcacagcaagtcacacaa

SEQ ID NO: 132 - DNA, 23 bp, Artificial
  Synthetic 800 AP50_7427_REV
atatggtgagaagtcgtgcgttt

SEQ ID NO: 133 - DNA, 32 bp, Artificial
  Synthetic 773 AP50_reverse PCR_left_end
ttggaatgttacagttatgttacagaaaaccg

SEQ ID NO: 134 - DNA, 62 bp, Artificial
  AP50 bacteriophage promoter
acttaccaattttagaaaatttcctattggaaagggtggaaaagtgtgacattattttttac